(19) United States
(12) Patent Application Publication
(10) Pub. No.: US 2003/0028904 A1
(43) Pub. Date: Feb. 6, 2003

(54) Genes involved in engulfment of dying cells and cell migration

(76) Inventors: Tina L. Gumienny, Piscataway, NJ (US); Enrico Brugnera, Warrenton, VA (US); Annie-Carole Tosello-Trampont, Charlottesville, VA (US); Jason M. Kinchen, Zurich (CH); Michael O. Hengartner, Zurich (CH); Kodimangalam S. Ravichandran, Charlottesville, VA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 09/881,579

(22) Filed: Jun. 14, 2001

Related U.S. Application Data
(60) Provisional application No. 60/285,469, filed on Apr. 19, 2001.

(51) Int. Cl. G01N 33/567; A01K 67/033; C07H 21/04; A61K 48/00; C12N 5/06

(52) U.S. Cl. 800/8; 800/3; 514/44; 435/7.21; 435/325; 435/320.1; 536/23.2

(57) ABSTRACT

The present invention pertains to a novel gene, CED-12, and its homologues, ELMO1, ELMO2 and ELMO3. The present invention also includes polypeptides that are encoded by these genes that are involved in the engulfment of dying cells and cell migration. The present invention also includes antibodies, assays, and treatment methods.

### RELATED APPLICATION

This application claims the benefit of U.S. Provisional Application No. 60/285,469, filed on Apr. 19, 2001, entitled “CED-12/ELMO, A Novel Member of the CRKII/DOCK180/RAC Pathway, Is Required For Phagocytosis and Cell Migration”. The entire teachings of the above application are incorporated herein by reference.

### GOVERNMENT SUPPORT

The invention was supported, in whole or in part, by Grant Nos. NIH-GM52540 and NIH-GM63310 from the National Institutes of Health. The Government has certain rights in the invention.

### BACKGROUND OF THE INVENTION

Some cells are programmed to die, while others die from a necrotic death. Phagocytosis is a process in which these cells or particles of dead cells are engulfed. Understanding the process of eliminating dead cells or particles of dead cells is important to various cellular mechanisms and the mechanism underlying various diseases states. Accordingly, a need exists to characterize genes involved in phagocytosis.

### SUMMARY OF THE INVENTION

The present invention relates to an isolated nucleic acid molecule having a nucleic acid sequence having at least about 60% (e.g., 70%, 75%, 80%, 85%, 90%, 95%) identity with SEQ ID NO.: 1, 3, 5, 7, 9, 11 or 13, or the coding region thereof; and encodes a polypeptide that modulates engulfment of dying cells or particles from dying cells, and cell migration. In particular, the present invention embodies an isolated nucleic acid molecule having a nucleic acid sequence of SEQ ID Nos.: 1, 3, 5, 7, 9, 11 or 13; the coding region of SEQ ID Nos.: 1, 3, 5, 7, 9, 11 or 13; the complement of a nucleic acid sequence of SEQ ID Nos.: 1, 3, 5, 7, 9, 11 or 13; the complement of a nucleic acid sequence of the coding region of SEQ ID Nos.: 1, 3, 5, 7, 9, 11 or 13; or a nucleic acid sequence that encodes SEQ ID Nos. 2, 4, 6, 8, 10, 12, 14 or 15. Additionally, the present invention pertains to nucleic acid molecules having a nucleic acid sequence that hybridizes under high stringency conditions to the nucleic acid sequence of SEQ ID Nos.: 1, 3, 5, 7, 9, 11 or 13; or the coding region of SEQ ID Nos.: 1, 3, 5, 7, 9, 11 or 13.

In one embodiment, the present invention relates to a probe containing the nucleic acid sequence that hybridizes under high stringency conditions to the nucleic acid sequence of SEQ ID Nos.: 1, 3, 5, 7, 9, 11 or 13; the coding region of SEQ ID Nos.: 1,3,5,7,9, 11 or 13.

The present invention embodies a polypeptide molecule having an amino acid sequence having at least about 60% (e.g., 70%, 75%, 80%, 85%, 90%, 95%) similarity with SEQ ID NO.: 2, 4, 6, 8, 10, 12, 14 or 15, or the coding region thereof and modulates engulfment of dying cells or particles of dying cells and cell migration. In particular, the present invention relates to a polypeptide molecule having an amino acid sequence of SEQ ID Nos.: 2, 4, 6, 8, 10, 12, 14 or 15; the coding region of SEQ ID Nos.: 2, 4, 6, 8, 10, 12, 14 or 15; or encoded by SEQ ID Nos.: 1, 3, 5, 7, 9, 11 or 13.

The present invention also embodies a vector or plasmid containing a nucleic acid sequence of SEQ ID Nos.: 1, 3, 5, 7, 9, 11 or 13; the coding region of SEQ ID Nos.: 1, 3, 5, 7, 9, 11 or 13; a complement of SEQ ID Nos.: 1, 3, 5, 7, 9, 11 or 13; a complement of the coding region of SEQ ID Nos.: 1, 3, 5, 7, 9, 11 or 13; or a nucleic acid sequence that encodes SEQ ID Nos. 2, 4, 6, 8, 10, 12, 14 or 15. In another embodiment, the present invention pertains to a vector or plasmid having a nucleic acid sequence that hybridizes under high stringency conditions to the nucleic acid sequence of SEQ ID Nos.: 1, 3, 5, 7, 9, 11 or 13 or the coding region thereof. Additionally, a vector or plasmid can contain a nucleic acid molecule that encodes an amino acid sequence of SEQ ID Nos.: 2, 4, 6, 8, 10, 12, 14 or 15; the coding region of SEQ ID Nos.: 2, 4, 6, 8, 10, 12, 14 or 15; or amino acid sequence encoded by SEQ ID Nos.: 1, 3, 5, 7, 9, 11 or 13.

The present invention includes, in an embodiment, a cell (e.g., a host cell) containing the nucleic acid sequence of SEQ ID Nos.: 1, 3, 5, 7, 9,11 or 13; the coding region of SEQ ID Nos.: 1, 3, 5, 7, 9, 11 or 13; a complement of a nucleic acid sequence of SEQ ID Nos.: 1, 3, 5, 7, 9, 11 or 13; a complement of a nucleic acid sequence of the coding region of SEQ ID Nos.: 1, 3, 5, 7, 9, 11 or 13; or a nucleic acid sequence that encodes SEQ ID Nos. 2, 4, 6, 8, 10, 12, 14 or 15. In another embodiment, the present invention relates to a cell having a nucleic acid sequence that hybridizes under high stringency conditions to the nucleic acid sequence of SEQ ID Nos.: 1, 3, 5, 7, 9, 11 or 13 or the coding region thereof. Also, the present invention pertains to a cell that contains a nucleic acid molecule that encodes an amino acid sequence of SEQ ID Nos.: 2, 4, 6, 8, 10, 12, 14 or 15; the coding region of SEQ ID Nos.: 2, 4, 6, 8, 10, 12, 14 or 15; or the amino acid sequence encoded by SEQ ID Nos.: 1, 3, 5, 7, 9, 11 or 13.

Yet another embodiment of the present invention, encompasses an antibody or antibody fragment (e.g., polyclonal, monoclonal, humanized or chimerized) that binds to a portion of a polypeptide molecule having an amino acid sequence of SEQ ID Nos.: 2, 4, 6, 8, 10, 12, 14 or 15; the coding region of SEQ ID Nos.: 2, 4, 6, 8, 10, 12, 14 or 15; or the amino acid sequence encoded by SEQ ID Nos.: 1, 3, 5, 7, 9, 11 or 13. The present invention also includes a fusion protein having the polypeptide described herein, and a portion of an immunoglobulin. An antagonist or agonist of a polypeptide molecule or nucleotide molecule, described herein, are encompassed by the present invention.

The present invention also includes a non-human transgenic animal having an isolated nucleic acid molecule or polypeptide molecule, as described herein. In particular, the present invention embodies a transgenic nematode worm containing a nucleic acid molecule having a nucleic acid sequence of SEQ ID NO.: 1, 3, 5, 7, 9, 11 or 13, wherein SEQ ID NO.: 1, 3, 5, 7, 9, 11 or 13 is mutated, deleted or encodes a non-functional polypeptide.

The present invention also relates to assays. In a particular embodiment, the present invention pertains to an assay for determining the presence or absence of a polypeptide molecule having an amino acid sequence of SEQ ID Nos: 2, 4, 6, 8, 10, 12, 14, or 15, in a sample. The assay involves obtaining the sample to be tested; contacting said sample with an antibody specific to a polypeptide molecule having an amino acid sequences of SEQ ID Nos: 2, 4, 6, 8, 10, 12, 14, or 15, or a fragment thereof, sufficiently to allow formation of a complex between the polypeptide and the antibody, and detecting the presence or absence of the complex formation. The methods of the present invention also include methods for determining whether a compound is an enhancer or inhibitor of phagocytosis of dying cells or cell migration. This embodiment involves exposing a transgenic nematode worm, as described herein, or transfected mammalian cell lines in the presences of dying cells to the compound to be tested, and measuring the level of phagocytic activity or cell migration, wherein an increase in the level of phagocytic activity or cell migration indicates an enhancer, and a decrease in the level of phagocytic activity or cell migration indicates an inhibitor. The present invention also includes compounds identified by these assays.

The present invention also includes methods of inhibiting phagocytosis of dying cells or cell migration in an organism, comprising subjecting the organism to a compound (e.g., an antibody or fragment thereof) that inhibits the polypeptide molecule, as described herein, wherein a decrease of phagocytic activity or cell migration occurs. The present invention further encompasses methods of enhancing phagocytosis of dying cells or cell migration in an organism, comprising subjecting the organism to a compound that inhibits the polypeptide molecule, as described herein, wherein an increase of phagocytic activity or cell migration occurs.

Additionally, the present invention encompasses therapeutic methods. In particular, the present invention includes methods of treating in mammals having a disease involving a defect in ELMO1, ELMO2 or ELMO3, or a pathway thereof, by administering a compound that contains the polypeptide molecule, as described herein, the nucleic acid molecule, as described herein, an antagonist thereof or an agonist thereof. The disease states where an increased phagocytic activity would be beneficial include inflammation, autoimmune disease and cancer. Disease states where a decreased phagocytic activity would be beneficial include neurodegenerative disease, stroke or sickle cell anemia.

Advantageously, the present invention includes a novel gene that is involved in the engulfment of dying cells and in cell migration. As a result, insight and understanding can be gained in the treatment of various diseases such as cancer, inflammation, autoimmune diseases, neurodegenerative diseases, stroke or sickle cell anemia.

### BRIEF DESCRIPTION OF THE DRAWINGS

FIG. 1 depicts the cDNA sequence of Caenorhabditis elegans CED-12 (SEQ ID NO: 1).

FIG. 2 depicts the amino acid sequence of C. elegans CED-12 (SEQ ID NO: 2).

FIG. 3 depicts the cDNA sequence of Drosophila CED-12 (SEQ ID NO: 3).

FIG. 4 depicts the amino acid sequence of Drosophila CED-12 (SEQ ID NO: 4).

FIGS. 5A-B depict the cDNA sequence of human ELMO1 (SEQ ID NO: 5).

FIG. 6 depicts the amino acid sequence of human ELMO1 (SEQ ID NO: 6).

FIGS. 7A-B depict the cDNA sequence of human ELMO2 (SEQ ID NO: 7).

FIG. 8 depicts the amino acid sequence of human ELMO2 (SEQ ID NO: 8).

FIG. 9 depicts the cDNA sequence of human ELMO3 (SEQ ID NO: 9).

FIG. 10 depicts the amino acid sequence of human ELMO3 (SEQ ID NO: 10).

FIGS. 11A-B depict the cDNA sequence of mouse ELMO1 (SEQ ID NO: 11).

FIG. 12 depicts the amino acid sequence of mouse ELMO1 (SEQ ID NO: 12).

FIGS. 13A-B depict the cDNA sequence of mouse ELMO2 (SEQ ID NO: 13).

FIG. 14 depicts the amino acid sequence of mouse ELMO2 (SEQ ID NO: 14).

FIG. 15 depicts the amino acid sequence of mouse ELMO3 (SEQ ID NO: 15).

FIG. 16 is a graphical representation of persistent cell corpses of the wild type and the following mutants: CED-12 (oz167), CED-12 (bz187), oz167/bz187, CED-12/CED-3, CED-2, CED-12/CED2, CED-5, CED-12/CED-5, CED-10, CED-12/CED-10, CED-1, CED-12/CED-1, CED-6, CED-12/CED-12, CED-7, CED-12/CED-7. Animals were scored for persistent cell corpses in the heads of young L1 larvae. Black bars, CED-12 single mutants; open bars, single engulfment mutants; gray bars, double mutant pairs for CED-12(oz167) and the other engulfment genes. Data shown are means ±standard deviation. *, p<0.01; **p<0.001 for double mutant versus the stronger of the respective single mutants.

FIGS. 17A-D show the CED-12 positional cloning strategy. FIG. 17A is a genetic map around the CED-12 locus. Genetic crosses placed CED-12 close to the left of lin-11 on chromosome I. FIG. 17B depicts a physical map of the CED-12 region. The YACs and cosmids shown were tested for their ability to rescue the engulfment defect of CED-12 mutants. All three YACs (bold) rescued CED-12 mutant animals, whereas the cosmids, either singly or in groups, failed to rescue. FIG. 17C is a schematic showing SNP mapping placed CED-12(oz167) within a 7 kb region in the gap between cosmids C25A1 and ZC247. This region, flanked by the polymorphisms opP19 and opP20, contains three predicted genes: Y106G6E.3, Y106G6E.4, and Y106G6E.5. Arrows indicate direction of transcription. FIG. 17D is a schematic showing the deduced structure of the Y106G6E.5, based on cDNA sequencing and RT-PCR. Open boxes, coding sequences. Thick arrow, 3′ UTR. Y1 06G6E.5 is trans-spliced to both SL1 and SL2 (SL in figure). Position and nature of characterized mutations are indicated.

FIG. 18A-B are schematics illustrating the identification and characterization of CED-12 homologues. FIG. 18A depicts the alignment of the predicted protein sequence of C. elegans CED-12, Drosophila CED-12, and ELMO1 and ELMO2 from mouse and human. Invariant residues are highlighted and conserved residues are shaded. The putative SH3 binding proline-rich motif in the C-terminus, present in all of the CED-12 family members, is underlined. The double underline around amino acid 630 represents the leucine zipper motif that is seen in both ELMO1 and ELMO2 of human and mouse, but is absent in the worm and fly proteins. Mutations affecting the C. elegans CED-12 protein are indicated by arrowheads above the worm protein sequence. FIG. 18C is a schematic diagram of the wilde type (wt) and the truncated forms of ELMO1, as well as Dock180 and CrkII constructs used in this study.

FIGS. 19A-D are graphical representations showing the effect of ELMO1 expression on phagocytosis and its synergy with Dock 180. FIG. 19A is a graphical representation depicting the inhibition of phagocytosis by transient expression of ELMO 1 or ELMO2. FIG. 19A shows the relative fluorescence intensity and the relative cell number for GFP, ELMO1 and ELMO2 transfectants. FIG. 19A also contains a histogram of the percent (%) of GFP positive cells with engulfed particles for GFP, ELMO1 and ELMO2 transfectants. The average Mean Fluorescence Intensity (MFI) values of duplicate samples calculated from such histograms are indicated on the top of the bar graphs. When the error bars are not visible, they were too small to be apparent. FIG. 19B is a histogram showing the percent (%) of GFP positive cells with engulfed particles for GFP, ELMO1+GFP, ELMO1+GFP+Rac1L61, Rac1L61, ELMO1+GFP+Rac1N17, Rac1N17, ELMO 1+GFP+CrkII, and CrkII transfectants. FIG. 19C shows the relative fluorescence intensity and the relative cell number for GFP, ELMO1, DOCK180, and ELMO1+DOCK180 transfectants. FIG. 19C also contains a histogram of the percent (%) of GFP positive cells with engulfed particles for GFP, ELMO1+GFP, DOCK180, ELMO1+GFP+DOCK180, DOCK180 de1GS, ELMO1+GFP+DOCK180 de1GS, DOCK180 de1PS, and ELMO1+GFP+DOCK180 de1PS transfectants. The numbers on the top of the bar graphs represent the average MFI values from duplicate samples. These data are representative of at least three independent experiments. FIG. 19D is a graphic representation of percent (%) of GFP positive cells with engulfed particles for GFP, GFP+ELMO−FLAG, GFP+ELMO−CAXX, GFP+DOCK180, GFP+DOCK180+ELMO−CAXX, GFP+ELMO+DOCK180, and GFP+ELMO−CAXX+DOCK180 transfectants. The average MFI values from duplicate samples are also indicated. Data are representative of two independent experiments.

FIGS. 20A-E are photographs of blots showing the biochemical interaction between ELMO1, Dock180 and CrkII. FIG. 20A is a photograph of a blot showing that ELMO1-GFP binds Dock180. COS-7 cells were transfected with 5 μg of ELMO1-GFP and His-tagged Dock180 plasmids either alone or together, using Superfect (Qiagen). 48hours post transfection, the cells were lysed and immunoprecipitated using anti-His antibodies and immunoblotted with anti-GFP or anti-His antibodies. The total lysates from the same experiment were run separately and immunoblotted with anti-GFP antibodies to determine ELMO1-GFP (100 kDa) expression. FIG. 20B is a photograph of a blot showing that N-terminal region of Dock180 is required for interaction with ELMO1. COS-7 cells were transfected with the indicated plasmids and the lysates were immunoprecipitated with anti-FLAG antibody and immunoblotted with anti-Dock180 (a mixture of N- and C-terminal antibodies). The same blot was stripped and reprobed with anti-FLAG to determine expression of the FLAG-tagged proteins. Immunoblotting of total lysates indicated the expression of Dock180 in the appropriate lanes. FIG. 20C is a photograph of a blot showing that ELMO1 and ELMO2 interact with Dock180. COS-7 cells were transfected as above with plasmids coding for FLAG-tagged ELMO1, ELMO2, the N-term or C-term versions of ELMO1 along with His-Dock180. After anti-FLAG immunoprecipitation, the coprecipitation of Dock180 was analyzed by immunoblotting. FIG. 20D is a photograph of a blot showing that CED-12 interacts with CED-5, but not Rac in a yeast two-hybrid assay. Interaction between Caenorhabditis elegans CED-12 and CED-5 as well as Rac wt and mutants were analyzed by yeast two-hybrid interaction as described in the Experimental procedures. Growth seen on selective plates is shown. FIG. 20E is a photograph of a blot showing that formation of a trimeric complex between ELMO1, CrkII and Dock180. After transient transfection of 293T cells with the indicated plasmids, the lysates were immunoprecipitated with anti-FLAG antibody and immunoblotted with anti-CrkII, anti-FLAG or anti-Dock180. Immunoblotting of the total lysates indicated the expression of the proteins in the appropriate lanes. A faint detection of endogenous Dock180 coprecipitating with ELMO1 (lanes 2 and 5) was observed.

FIGS. 21A-C are graphical representations showing that ELMO1/Dock180/CrkII signal through Rac to affect engulfment. FIG. 21A is a histogram showing the functional cooperation between ELMO1, Dock 180 and CrkII. FIG. 21A shows the percent (%) of GFP positive cells with engulfed particles for GFP, ELMO1, DOCK180, ELMO1+DOCK180, CrkII, ELMO1+CrkII, DOCK180+CrkII, ELMO1+DOCK180+CrkII, ELMO1+DOCK180 de1PS+CrkII, and ELMO1+DOCK180 de1GS+CrkII transfectants. LR73 cells were analyzed in a phagocytosis assay (as described in FIG. 19 above), after transient transfection with ELMO1−GFP (0.7 μg), Dock180 (1.5 μg), CrkII (0.4 μg), de1PSDock180 (1.2 μg) or de1GSDock180 (0.8 μg). Carrier DNA was added to keep the same plasmid concentration in the different conditions. The average MFI value of duplicate samples is shown at the top of the bar graphs. These data (as well as data shown in FIGS. 21B and C) are representative of at least three independent experiments. FIG. 21B shows that SH3 domain mutant of CrkII fails to synergize with ELMO1 and Dock180. FIG. 21B shows the percent (%) of GFP positive cells with engulfed particles for GFP, ELMO1, DOCK180, ELMO1+DOCK180, ELMO1+DOCK180+CrkII, and ELMO1+DOCK180+CrkII W169L transfectants. LR73 cells were transiently transfected with ELMO1-GFP (0.7 μg), Dock180 (1.5 μg), and either the wild type or mutant CrkII (0.4 μg), and the ELMO1-GFP positive cells were analyzed for phagocytosis. FIG. 21C shows that enhanced uptake due to CrkII+ELMO1 or Dock180+ELMO1 coexpression is inhibited by Rac1N17. FIG. 21C shows the percent (%) of GFP positive cells with engulfed particles for GFP, ELMO1+GFP, ELMO1+GFP+DOCK180, ELMO1+GFP+DOCK180+CrkII, ELMO1+GFP+DOCK180+RacN17 and ELMO1+GFP+DOCK180+CrkII+RacN17 transfectants. LR73 cells were transiently transfected with ELMO1-GFP (0.6 μg), Dock180 (1 μg), CrkII (0.4 μg) and 1.5 μg of Rac1N17. The GFP positive cells were analyzed for phagocytosis. The expression of all of the transfected proteins was confirmed by immunoblotting for the individual proteins in total lysates taken from parallel samples.

FIG. 22 is a schematic for ELMO1 involvement in Rac activation.

### DETAILED DESCRIPTION OF THE INVENTION

The Structure of CED-12, ELMO1, ELMO2 and ELMO3

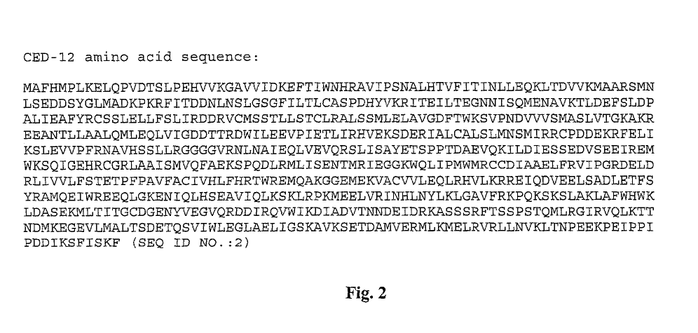

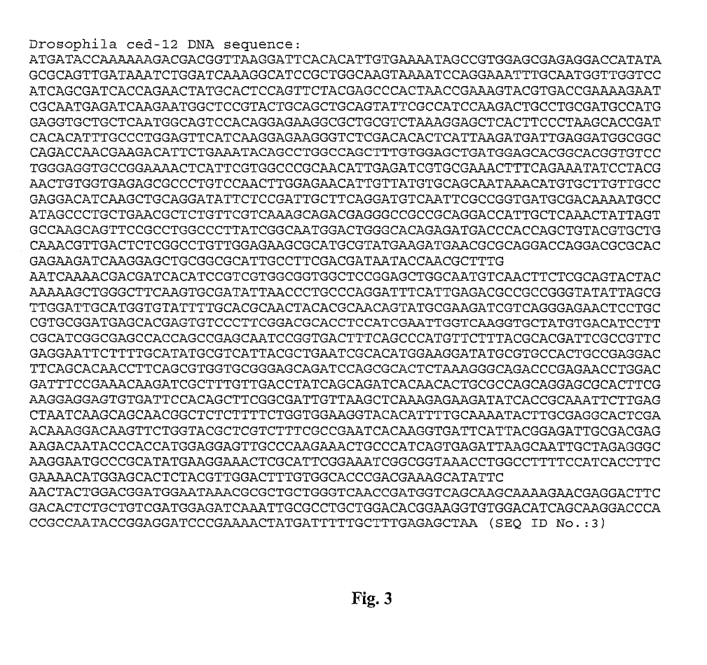

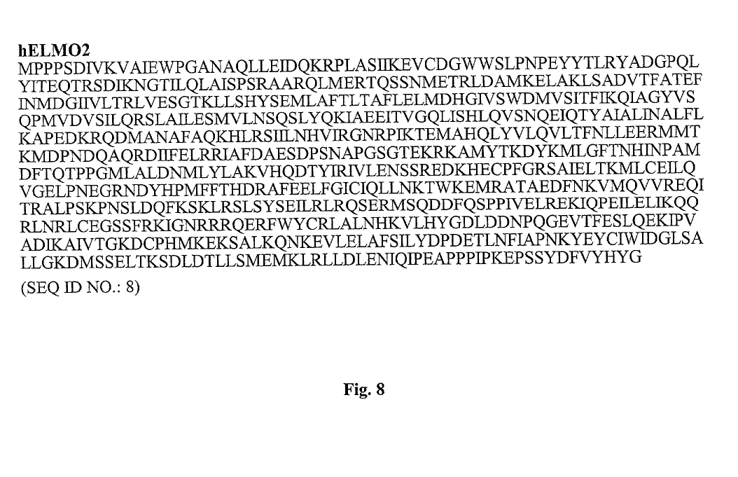

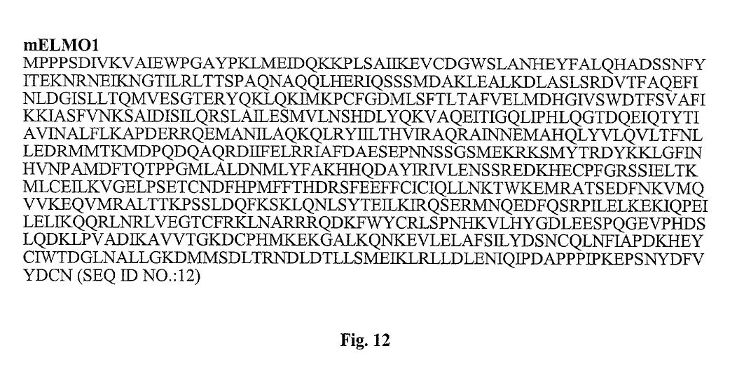

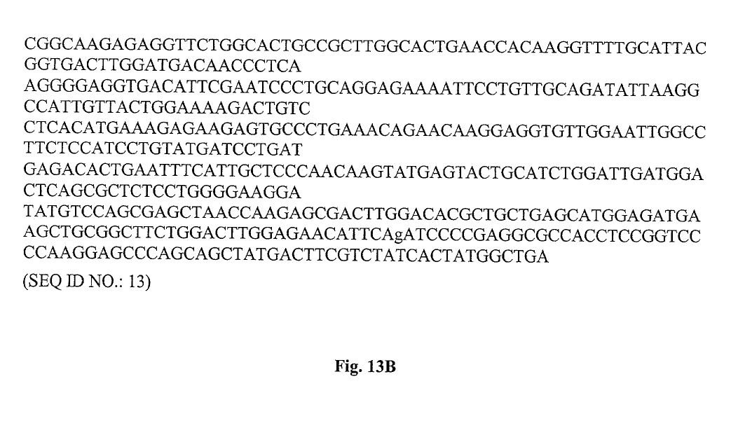

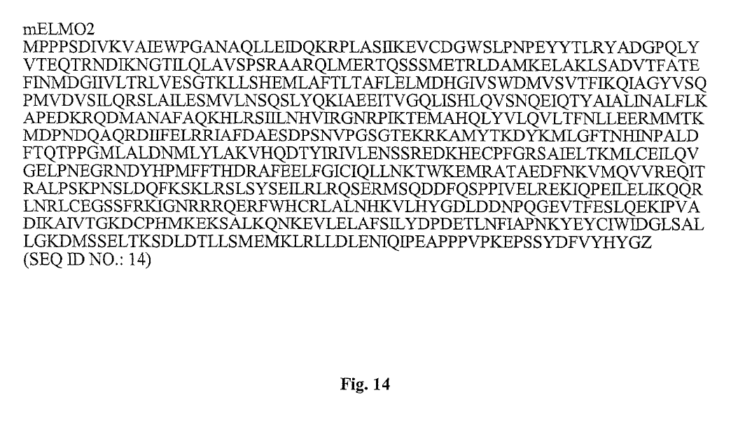

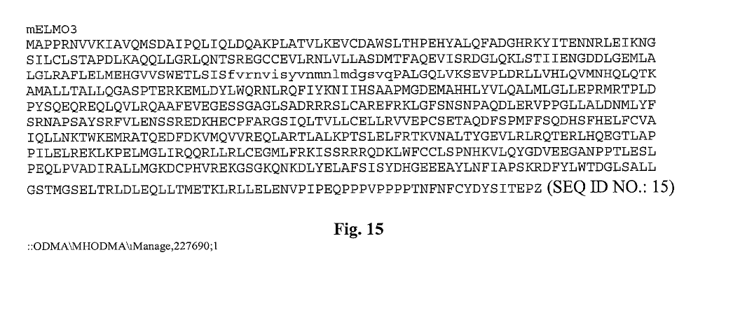

The present invention relates to newly discovered genes, CED-12, ELMO1, ELMO2 and ELMO3 that modulate phagocytosis of dying cells and migration of cells. The invention encompasses proteins and genes from various species. In particular, the protein of the present invention has been isolated in nematodes (i.e., C. elegans) (SEQ ID NO.: 2), Drosophila (SEQ ID NO.:4), human (SEQ ID Nos. 6, 8, and 10), and mouse (SEQ ID NO.: 12, 14 and 15) species. See FIGS. 2, 4, 6, 8, 10, 12, 14, and 15. In particular, three human homologues of CED-12 have been identified and isolated: ELMO1 (SEQ ID NO.: 6), ELMO2 (SEQ ID NO.: 8) and ELMO3 (SEQ ID NO.: 10); three murine homologues were isolated: ELMO1 (SEQ ID NO.: 12), ELMO2 (SEQ ID NO.: 14) and ELMO3 (SEQ ID NO.: 15); and a Drosophila homologue was also identified: CED-12 (SEQ ID NO.:4). Each of the human homolgues is approximately 44% similar to the C. elegans CED-12 amino acid sequence, and 65% similar to Drosophila CED-12 amino acid sequence. When human ELMO1 and human ELMO2 were compared, there was a 75% identity and 88% similarity.

The invention also encompasses the full length ELMO1, ELMO2, ELMO3 and CED-12 proteins (SEQ ID NO.: 2, 4, 6, 8, 10, 12, 14, or 15) and functional portions thereof. The protein has several functional domains or portions. For example, the proteins have a N-terminus and a C-terminus. The ELMO1, ELMO2, ELMO3 and CED-12 proteins also have a SH3 binding PxxP motif near the C-terminus, and ELMO1, ELMO2 and ELMO3 proteins also have a putative leucine zipper at approximately amino acid number 630. Therefore, the present invention not only includes the full length ELMO1, ELMO2, ELMO3 and CED-12 proteins, but the various functional portions thereof In particular, the present invention relates to the N-terminus, the C-terminus, the SH3 binding PxxP domain, the leucine zipper domain, and functional combinations thereof.

The Function of ELMO1, ELMO2, and CED-12 proteins

The engulfment or the removal of cells undergoing apoptosis (e.g., programed cell death) is the final step of the apoptotic program. Genetic screens in the nematode, Caenorhabditis elegans, have led to the identification of over a dozen genes that function in the apoptotic program. Seven of these genes, CED-1, CED-2, CED-5, CED-6, CED-7, CED-10 and now CED-12 are involved in the efficient engulfment of apoptotic cells. In worms that are mutant for any of these seven genes, many cell corpses fail to be engulfed and linger in the body for hours or even days. Double mutant analyses have placed six of these engulfment genes into two partially redundant functional pathways: the CED-1/CED-6/CED-7 pathway and the CED-2/CED-5/CED-10 pathway. As described herein, CED-12 is part of the second pathway, the CED-2/CED-5/CED-10 pathway.

These six genes have been cloned in the worm and their homologues identified in higher organisms. Within the first group, the CED-1 gene encodes a transmembrane protein (with homology to the mammalian acetylated LDL receptors), CED-7 encodes an ATP binding cassette transporter (with homology to mammalian ABC1 and ABCR), and CED-6 encodes an evolutionarily conserved candidate adapter protein. CED-1 and CED-7 proteins (and their homologues in mammals) participate in the recognition of dying cells and recruit/activate intracellular signaling molecules, at least one of which may be CED-6, to facilitate engulfment.

In the second group of genes, CED-2 encodes a protein homologous to the mammalian adapter protein CrkII, CED-5 encodes the homologue of the mammalian Dock180 protein, and CED-10 encodes a homologue of the Rac1 GTPase. The CED-2/CrkII adapter protein is composed of one Src-homology 2 (SH2) and two Src-homology 3 (SH3) domains. CrkII was originally identified as part of a viral oncogene, but has since been implicated in a variety of cell shape and cytoskeletal changes. Dock180 binds to the first SH3 domain of CrkII, and overexpression of a membrane-targeted Dock 180 led to cytoskeletal changes in NIH3T3 cells.

In mammalian cells, the Rac GTPase has been implicated in actin polymerization at the edges of the cell, the formation of lamellipodia and cell motility, and in Fc receptor-mediated phagocytosis. Dock180 is thought to function upstream of Rac since it can directly bind the nucleotide-free form of Racl (but not Rho or cdc42) and promote the formation of the GTP-bound activated form of Rac. The Drosophila Dock180 homologue, Myoblast city (Mbc) has also been identified as an upstream regulator of Rac1. Genetic studies in C. elegans have also placed CED-10/Rac downstream of CED-2/CrkII and CED-5/Dock180. Thus, it has been proposed that the second complementation group of genes plays a role in reorganization of the cytoskeleton during engulfment. Recent studies in mammalian cells have confirmed that CrkII and Rac can regulate the engulfment of apoptotic cells.

In addition to the defect in engulfment of dying cells, worms with mutations in CED-2, CED-5, or CED-10 also show abnormal migration of the distal tip cells (DTC). CED-10/Rac1 localizes to cell membranes during larval elongation, another process that requires extensive cell migration. Interestingly, in mammalian cells, CrkII, Dock180 and Rac1, along with another adapter protein p130Cas, have also been linked to cell migration, invasion of tumor cells, and cell survival. Thus, Rac1 and its upstream activators likely play a key role in cellular migration during development.

The present invention relates to CED-12 and its mammalian orthologues, ELMO1, ELMO2, and ELMO3 as novel members of the CrkII/Dock180/Rac pathway. In C. elegans, CED-12 is required for the efficient engulfment of dying cells and is also essential for certain cell migrations. In mammalian cells, ELMO1 functionally cooperates with CED-2/CrkII and CED-5/Dock180 during phagocytosis and functions upstream of Rac1. Biochemically, ELMO1 binds to Dock180 and also forms a ternary complex with Dock180 and CrkII. These studies place CED-12 or ELMO1, ELMO2 and ELMO3 as important members of the CED-2/CED-5/CED-10 or CrkII/Dock180/Rac pathway, respectively, that regulates engulfment and cell migration in C. elegans and mammals.

The ELMO1, ELMO2 and CED-12 polypeptides modulate (e.g., increase or decrease) phagocytosis of dying cells. Although experiments have not yet performed directly with ELMO3, based on the very high sequence homology to ELMO1 and ELMO2, it is reasonable to believe that ELMO3 will behave similarly in phaogycotosis. The process of phagocytosis refers to the engulfment of dying cells or particles of dying cells. The phagocytic process involves the partial or entire engulfment of dying cells or particles thereof. These dying cells or particles from dying cells include any cell or particle from a cell undergoing biological processes related to dying, and in particular those cells or particles from cells undergoing a necrotic death or an apoptotic death.

The ELMO1, ELMO2 and CED-12 polypeptides are also involved in cell migration and developmental processes. Although experiments have not yet performed directly with ELMO3, based on the very high sequence homology to ELMO1 and ELMO2, it is reasonable to believe that ELMO3 will behave similarly in phaogycotosis. Cell migration refers to the movement of cells such as elongation and other cytoplasmic movement or partitioning, which occurs during the development of embryos. In particular, cell migration is part of developmental processes, processes involved with germ cell maturation, such as the formation of the embryo, vulval formation, fertility, brood size and growth, larval development, hatching, morphogenesis, and elongation. Additional processes of ELMO1, ELMO2, ELMO3 and CED-12 polypeptides are further described herein.

CED-12, a novel protein, has been isolated in C. elegans, as well as its orthologues in Drosophila, human and mouse. In the worm, the CED-12 gene product is needed within the engulfing cell for phagocytosis of early and late cell corpses, as well as for cell migrations in the embryo and the gonad. Studies with the mammalian orthologues ELMO1 and ELMO2 indicate that these proteins are also involved in phagocytosis and that they function in conjunction with two other known members of the engulfment pathway, CED-2/CrkII and CED-5/Dock180. Moreover, during phagocytosis, ELMO1 functions upstream of CED-10/Rac1, the fourth known member of this pathway in C. elegans and mammals. Additionally, expression of ELMO1 and ELMO2 in CED-12 deficient worms rescues the distal tip cell migration defect and overexpression of ELMO1 results in Rac1-dependent cytoskeletal changes. Since mutations in CED-12 cause a panoply of defects in the worm, CED-12 and its mammalian orthologues play a key role in integrating signals from a variety of stimuli to Rac dependent actin-based cytoskeletal rearrangements that regulate phagocytosis and cell migration.

Genetically, CED-2/CED-5/CED-10 and CED-1/CED-6/CED-7 segregate into two partially redundant groups. Analyzed CED-12 alleles showed a defect in both engulfment and cell migration, similar to those observed in CED-2, CED-5, and CED-10 mutant animals. See Examples. Double mutant analyses also indicate that CED-12 acts in the same pathway as the CED-10 pathway mutants. Even though all of the known engulfment genes have been cloned, the upstream activators of the CED-2/CED-5/CED-10/CED-12 pathway for engulfment remain elusive, since none of the four genes in this complementation group encode a receptor. Determining whether CED-1 and/or CED-7 cross-talks with the Rac pathway and defining the precise intersection point(s) between the two partially redundant pathways during engulfment awaits further investigation. In mammals, in addition to the homologues of CED-1 and CED-7, a number of receptors that participate in engulfment have been recognized, including some integrins that are known to function upstream of Crk. Whether one or more of these receptors signal through the CrkII/Dock180/ELMO/Rac pathway remains to be seen.

CED-12/ELMO and CED-5/Dock180 Connection

An evolutionary conserved interaction exists between CED-12/ELMO and CED-5/Dock180 in COS or 293T cell expression systems and in a yeast two hybrid assay. While the gross regions of interaction required in the two proteins were identified, the precise nature of their interaction is not known. Dock180 is a large protein of 1866 amino acids, and contains an SH3 domain at its extreme N-terminus and proline-rich motifs near its C-terminus, but no other functional domains or motifs have been characterized. A detailed mutational analysis of the interaction between ELMO and Dock180 yields clues on how these proteins regulate each other and thereby Rac. Although an interaction between ELMO and Dock180 could be readily detected when overexpressed in COS or 293T cells, it is not known whether their interaction occurs at endogenous levels of expression and whether it requires receptor-mediated signals. So far, the anti-peptide antibodies generated against ELMO have not been of high enough affinity to address this question using endogenous proteins. The precise reason for partial inhibition of phagocytosis seen with either ELMO alone or Dock180 alone expression remains unclear; however, the enhancement of uptake seen when the two proteins are expressed, while transfection with either one alone was inhibitory, suggests that perhaps the ELMO/Dock180 complex needs to be at a certain optimum level, and that overexpression of one can affect the balance. A second isoform of Dock180, Dock-2, with a more restricted expression in hematopoietic cells, as well as other less-well characterized homologues of Dock180 have been described. The potential interaction of Dock-2 or other Dock180 homologues with ELMO1 and ELMO2 remains to be tested.

ELMO1, Dock180 and CrkII form a complex, with the data supporting Dock180 acting as a scaffold to which ELMO and CrkII bind. An ELMO1:Dock180 complex might exist initially, to which CrkII binds. This is based on the observations that ELMO1 binds to a mutant of Dock180 that lacks the CrkII binding sites, and CED-12 and CED-5 could interact in the absence of CED-2 in yeast two-hybrid assays (there is no Crk II homologue in S. cerevisiae). However, CrkII could still strengthen the ELMO:Dock180 interaction in a trimeric complex. At present, the stoichiometry of each of these proteins is unknown. In the phagocytosis assays, the most efficient uptake was seen when all three proteins were cotransfected. While a synergy was seen after cotransfection of ELMO1 and CrkII without concurrent transfection of Dock180, this enhancement was perhaps mediated through the endogenous Dock180. In fact, mutants of CrkII that cannot interact with Dock180, or mutants of Dock180 that cannot bind to either ELMO or CrkII failed to show the same type of synergy in phagocytosis that was seen when the three wild type plasmids were cotransfected. These data suggest that Dock180 is the intermediary through which ELMO and CrkII biochemically and functionally interact.

CED-12/ELMO1 and Rac Activation

Several lines of evidence suggested that CED-12/ELMO functions upstream of Rac. (i) Previous genetic studies in the worm have indicated that CED-10/Rac functions downstream of CED-2/CrkII and CED-5/Dock180, while CED-2 and CED-5 function at a parallel level. Overexpression of CED-12 failed to rescue the engulfment defect in CED-2 or CED-5 deficient animals. (ii) In mammalian cells, the partial inhibition of uptake seen due to transfection of ELMO1 was overcome by expression of an activated form of Rac1; (iii) CrkII functions upstream of Rac1 in mammalian phagocytosis and the interaction of ELMO1 with Dock180 and CrkII suggests that ELMO1 would also function at a step upstream of Rac1; (iv) The enhanced uptake due to cotransfection of ELMO1 with Dock180 and/or CrkII was inhibited by a dominant negative form of Rac; (v) The cytoskeletal changes induced by overexpression of ELMO1/Dock180/CrkII were inhibited by a dominant negative Rac1, without an apparent effect on the membrane localization of ELMO1. Based on these, a model whereby ELMO, in conjunction with Dock180 and CrkII, would lead to Rac1 activation is favored.

Dock180 has been shown to bind nucleotide-free Rac, a conformation of Rac that appears to be favored by guanine nucleotide exchange factors (GEFs). Based on genetic studies in Drosophila and overexpression studies in NIII3T3 cells, Dock180 is thought to regulate the GTP-loading of Rac and lead to its activation. However, Dock180 itself does not possess an obvious GEF-like domain in its primary sequence and is thought to recruit a Rac-GEF. Although CED-12 itself might be the GEF, the sequence analyses failed to detect any Rac GEF domain in CED-12/ELMO polypeptides. Thus, it appears more likely that an existing or yet to be identified Rac-GEF activity is associated with the ELMO1/Dock180/CrkII complex and leads to Rac activation.

The fact that the DTC migration defect can still be observed in CED-12; CED-3 double mutants suggests that the engulfing cells and migrating cells receive different cues, with CED-12 and the other known Rac1 pathway members acting downstream of these specific signals. Several additional genes required for proper DTC migration have been described. Which ones, if any, of these genes might encode upstream regulators of CED-2/CED-5/CED-12/CED-10 remains to be determined. In the mammalian system, a role for CrkII and Dock180 along with p130Cas has been documented in integrin and Rac-1 dependent cell migration. The interaction of ELMO1 with Dock180 and CrkII suggests that ELMO1 would also play a role in mammalian cell migration. This is supported by the localization of ELMO1-GFP to the membrane ruffles that play a role in membrane protrusion during migration. Moreover, CED-12 mutant animals that are transgenic for the murine ELMO1 showed a partial rescue of the gonadal tip cell defect, suggesting a conserved role for CED-12/ELMO in cell migration. At present it is not clear whether the cytoskeletal changes and membrane protrusions during phagocytosis and cell migration are analogous. A further detailed analysis aimed at determining the function of ELMO1 and ELMO2 during mammalian cell migration can reveal similarities and differences compared to engulfment, and the specific function of CED-12/ELMO proteins in the two processes.

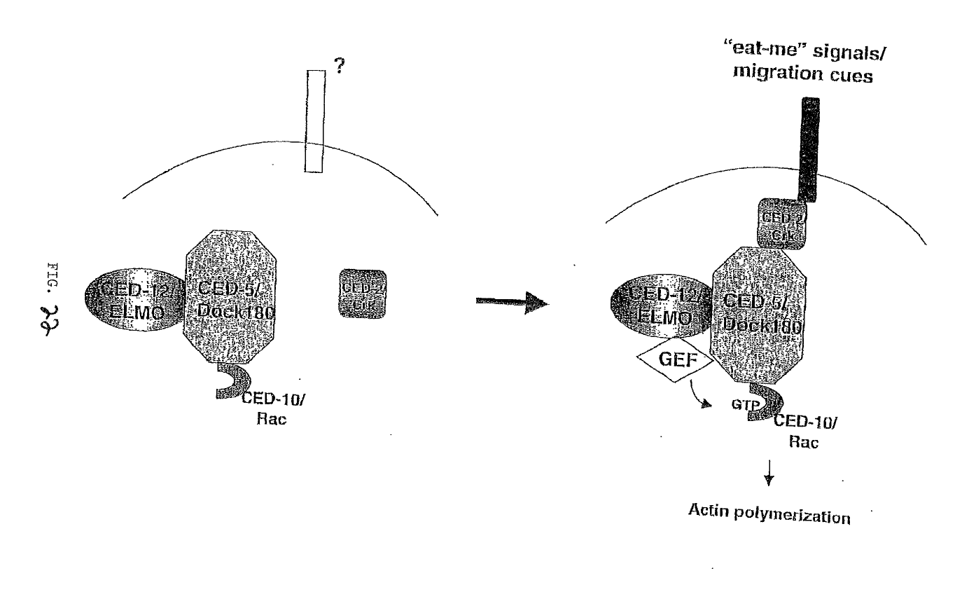

Based on the existing data, the working model for Rac activation through the ELMO1/Dock180/CrkII pathway is depicted in FIG. 22. ELMO1 and Dock180 likely exist basally in a complex without CrkII. Upon receiving engulfment signals or migration cues, appropriate membrane receptors would be activated and lead to formation of the ELMO/Dock180/CrkII complex as well as its localization. It is not known which of these three proteins mediates the recruitment. A Rac-GEF then binds to this complex and lead to activation of Rac that is bound to Dock180. ELMO1 could play a critical role, perhaps in recruiting the GEF or its activity toward Rac.

ELMO1 and Dock180 bind in a complex initially. The fluorescence microscopy data with overexpressed proteins suggests that this complex likely resides predominantly in the cytoplasm. Since CrkII is not generally found in complex with Dock180 under basal conditions, CrkII may not be part of the ELMO/Dock180 complex. Either the “eat-me” signals on apoptotic cells or migration cues would promote an interaction of CrkII with Dock180, and in turn, with ELMO. It is not known at present where the trimeric complex is formed and whether one or more than one of these three proteins mediates the interaction with a putative receptor(s). It is noteworthy that the ELMO1-CAAX construct did not provide a gain of function for ELMO1, and can reflect another level of regulation besides the simple membrane localization. Once assembled, the trimeric complex likely recruits a GEF and promotes the GTP:GDP exchange on the nucleotide-free Rac that is bound to Dock180. Given the various defects seen in CED-12 mutant worms, which suggest a critical role for this protein, it is likely that CED-12/ELMO itself recruits a Rac-GEF, or regulates the Dock180 dependent-nucleotide exchange on Rac in some other critical way. It is not known whether p130Cas, a protein that interacts with the Crk-SH2 domain and also implicated in cell migration, is also recruited to the CrkII/Dock180/ELMO complex and what potential effects it can have on this complex. Testing the validity of this model is currently in progress.

Forms of ELMO1, ELMO2, ELMO3 and CED-12 Polypeptides

The present invention is intended to encompass ELMO1, ELMO2, ELMO3 and CED-12 proteins, and proteins and polypeptides having amino acid sequences analogous to the amino acid sequences of the ELMO1, ELMO2, ELMO3 and CED-12 proteins and functional equivalents thereof. The terms “ELMO1”, “ELMO2”, ELMO3”, and “CED-12” refer to the ELMO1, ELMO2, ELMO3 and CED-12 polypeptides, respectively. The term “ELMO” refer to ELMO1, ELMO2 and ELMO3 collectively. Such polypeptides are defined herein as analogs (e.g., homologues) or derivatives. The present invention is also intended to embody the various functional domains, as described herein. Analogous amino acid sequences are defined herein to mean amino acid sequences with sufficient identity to the ELMO1, ELMO2, ELMO3 and CED-12 amino acid sequences so as to possess the biological activity of the native protein. For example, an analogous peptide can be produced with “silent” changes in amino acid sequence wherein one, or more, amino acid residues differ from the amino acid residues of the isolated protein, yet still possess at least one biological activity of ELMO1, ELMO2, ELMO3 and CED-12 proteins (e.g., promote the phagocytosis of dying cells, or cell migration). Examples of such differences include additions, deletions or substitutions of residues of the amino acid sequence. Also encompassed by the present invention are analogous polypeptides that exhibit greater, or lesser, biological activity of the protein of the present invention.

The “biological activity” of the ELMO1, ELMO2, ELMO3 and CED-12 proteins are defined herein to mean the ability to promote phagocytosis of dying cells (e.g., necrotic cells and/or apoptotic cells) and/or cell migration (e.g., developmental processes). In particular, ELMO1 functions in the CrkII/Dock180/Rac pathway. ELMO1 binds to DOCK180, to form a complex, which then binds with CrkII. The ELMO1/DOCK180/CrkII complex activates the Rac protein, and is a key element in the phagocytosis process of dying cells. ELMO2 also binds DOCK180 and functions as a member of the CrkII/Dock180/Rac pathway. Additionally, ELMO1 and ELMO2 are integrally related in the process of cell migration and the development processes, as defined herein. When ELMO1 and ELMO2 were transfected in C. elegans mutants having a non-functional CED-12, ELMO1 and ELMO2 were expressed and completely rescued the DTC migration defect, indicating that ELMO1 and ELMO2 interacts with CED-12 partners. See the Examples. The biological activity of ELMO1, ELMO2 and CED-12 proteins, and the predicted activity of ELMO3, are also defined by the various functional characteristics as described herein, and in particular, in the Examples.

Similarly, CED-12 possesses similar biological activity. In C. elegans, CED-2 is homologous of CrkII, CED-5 is homologous to DOC180, and CED-10 is homologous to Rac1. CED-12 functions in the CED-2/CED-5/CED-10 pathway. CED-12 acts upstream of CED-10 and activates CED-10. CED-12 is a key polypeptide that acts in the phagocytic process of dying cells. Additionally, CED-12 is involved the process of cell migration and the development processes, as defined herein. CED-12 plays a role in developmental processes including, for example, to size, formation, larval development, synchronized hatching, fertility, and cell migration (e.g., elongation process).

The present invention also encompasses biologically active polypeptide fragments of the ELMO1, ELMO2, ELMO3 and CED-12 polypeptides described herein. Such fragments can include only a part of a full length amino acid sequence of the ELMO1, ELMO2, ELMO3 and CED-12 polypeptides and yet possess the ability to either induce phagocytic function of dying cells, cell migration and/or developmental processes. For example, polypeptide fragments comprising deletion mutants of the ELMO1, ELMO2, ELMO3 and CED-12 polypeptides can be designed and expressed by well known laboratory methods. Such polypeptide fragments can be evaluated for biological activity as described herein.

Biologically active derivatives or analogs of the above described ELMO1, ELMO2, ELMO3 and CED-12 polypeptides, referred to herein as peptide mimetics can be designed and produced by techniques known to those of skill in the art. (see e.g., U.S. Pat. Nos. 4,612,132; 5,643,873 and 5,654,276). These mimetics can be based, for example, on a specific ELMO1, ELMO2, ELMO3 and CED-12 amino acid sequence and maintain the relative position in space of the corresponding amino acid sequence. These peptide mimetics possess biological activity similar to the biological activity of the corresponding peptide compound, but possess a “biological advantage” over the corresponding ELMO1, ELMO2, ELMO3 and CED-12 amino acid sequence with respect to one, or more, of the following properties: solubility, stability and susceptibility to hydrolysis and proteolysis.

Methods for preparing peptide mimetics include modifying the N-terminal amino group, the C-terminal carboxyl group, and/or changing one or more of the amino linkages in the peptide to a non-amino linkage. Two or more such modifications can be coupled in one peptide mimetic molecule. Modifications of peptides to produce peptide mimetics are described in U.S. Pat. Nos. 5,643,873 and 5,654,276. Other forms of the ELMO1, ELMO2, ELMO3 and CED-12 polypeptides, encompassed by the present invention, include those which are “functionally equivalent.” This term, as used herein, refers to any nucleic acid sequence and its encoded amino acid, which mimics the biological activity of the ELMO1, ELMO2, ELMO3 and CED-12 polypeptides and/or functional domains thereof.

The ELMO1, ELMO2, ELMO3 and CED-12 polypeptides can be in the form of a conjugate or a fusion protein, which can be manufactured by known methods. Fusion proteins can be manufactured according to known methods of recombinant DNA technology. For example, fusion proteins can be expressed from a nucleic acid molecule comprising sequences which code for a biologically active portion of the ELMO1, ELMO2, ELMO3 or CED-12 polypeptide and its fusion partner, for example a portion of an immunoglobulin molecule. For example, some embodiments can be produced by the intersection of a nucleic acid encoding immunoglobulin sequences into a suitable expression vector, phage vector, or other commercially available vectors. The resulting construct can be introduced into a suitable host cell for expression. Upon expression, the fusion proteins can be isolated or purified from a cell by means of an affinity matrix.

The ELMO1, ELMO2, ELMO3 or CED-12 protein and nucleic acid sequences include homologues (e.g., C. elegans, Drosophila, mouse or human) as defined herein. Hence, the present invention includes mammalian protein or nucleic acid sequences. The homologous proteins and nucleic acid sequences can be determined using methods known to those of skill in the art. Initial homology searches can be performed at NCBI against the GenBank, EMBL and SwissProt databases using, for example, the BLAST network service. Altschul, S. F., et al., J. Mol. Biol., 215:403 (1990), Altschul, S. F., Nucleic Acids Res., 25:3389-3402 (1998). Computer analysis of nucleotide sequences can be performed using the MOTIFS and the FindPatterns subroutines of the Genetics Computing Group (GCG, version 8.0) software. Protein and/or nucleotide comparisons were performed according to Higgins and Sharp (Higgins, D. G. and Sharp, P. M., Gene, 73:237-244 (1988) e.g., using default parameters). Homologous proteins and/or nucleic acid sequences of the present invention are defined as those molecules with greater than 45% sequence identity and/or similarity (e.g., greater than 50%, 60%, 70%, 75%, 80%, 85%, 90%, or 95% identity and/or similarity).

Forms of Nucleic Acid

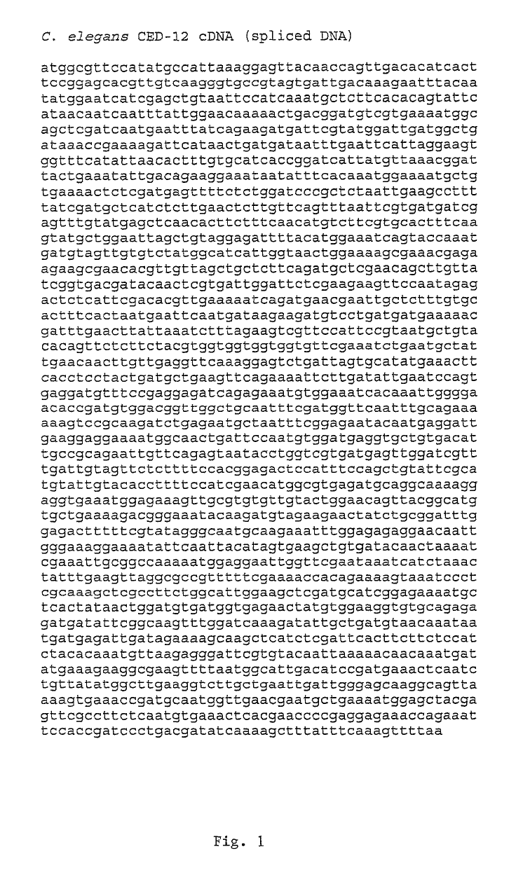

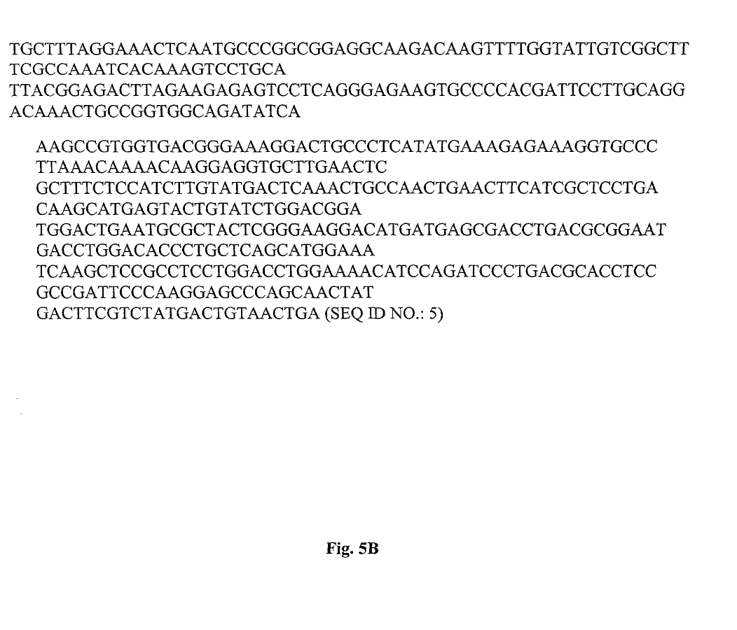

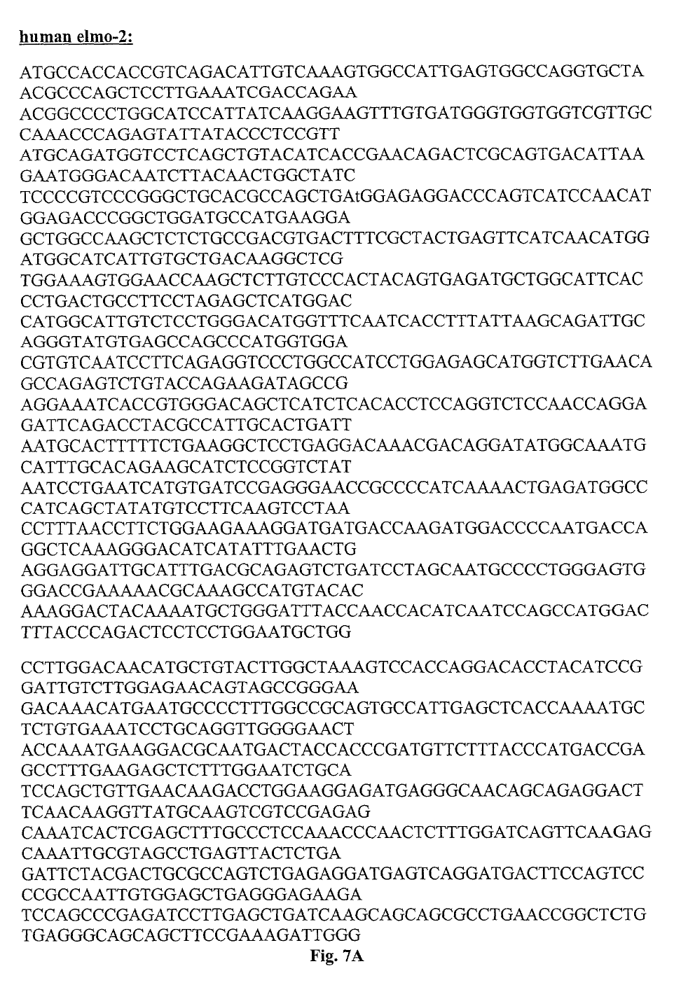

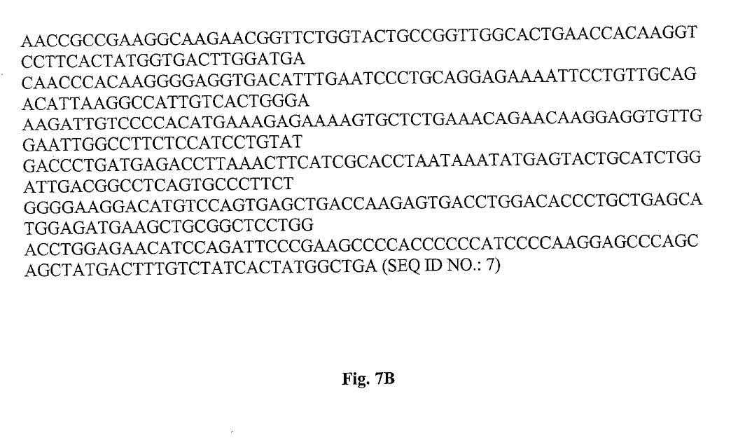

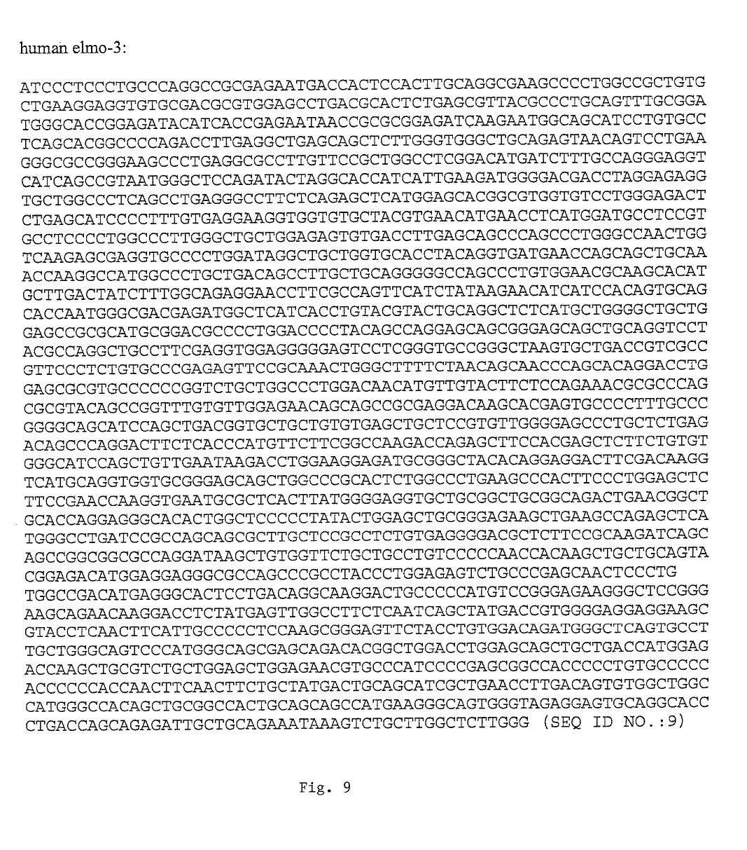

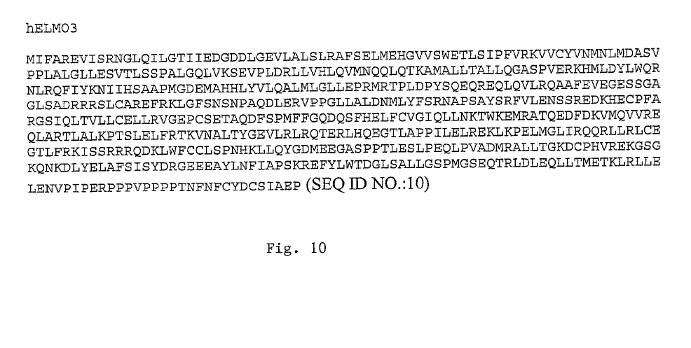

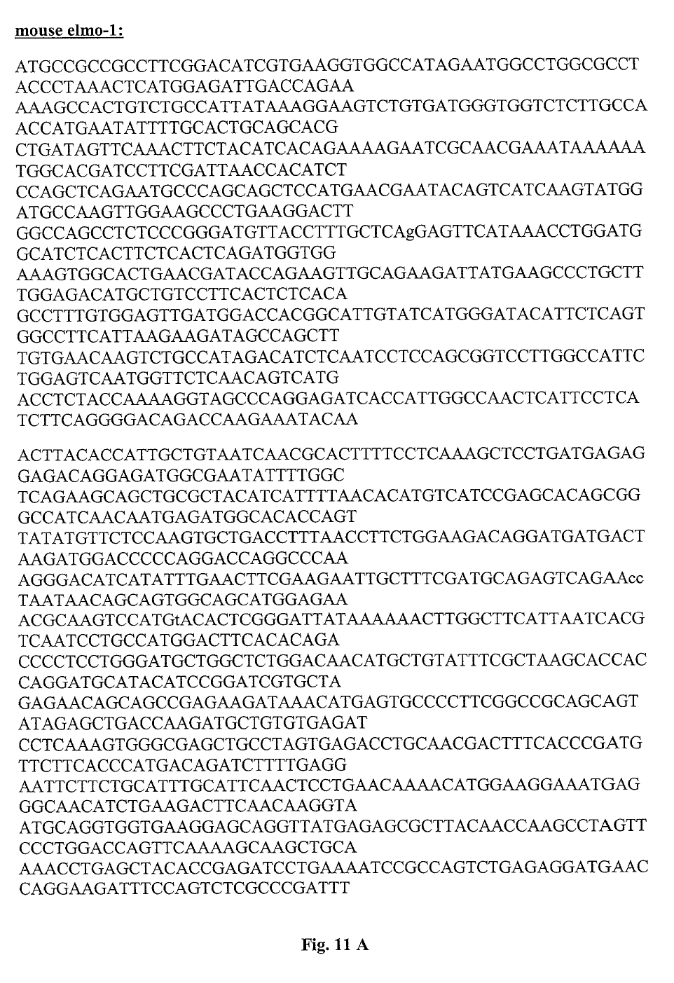

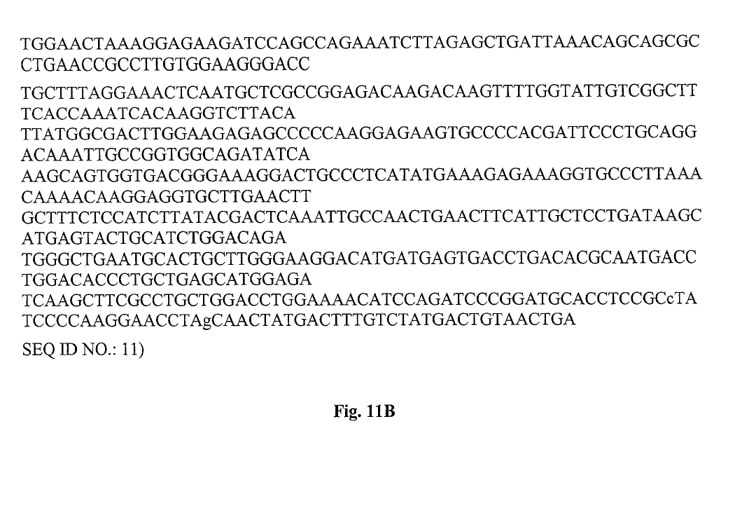

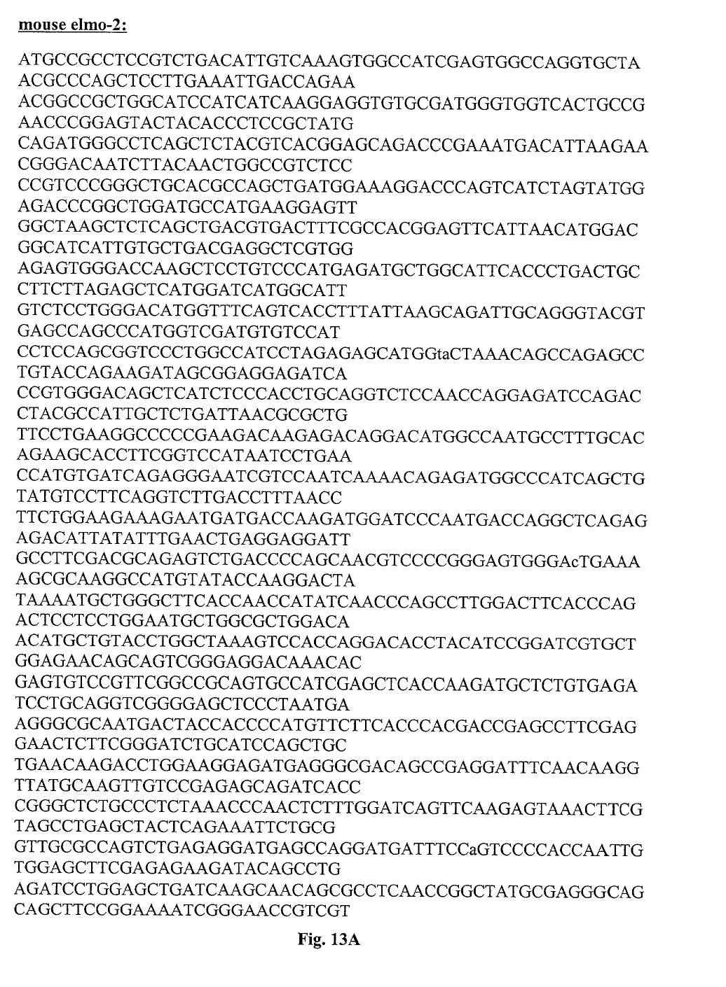

The present invention also encompasses isolated nucleic acid molecules that comprise sequences (SEQ ID Nos.: 1, 3, 5, 7, 9, 11 or 13) encoding the ELMO1, ELMO2, ELMO3 or CED-12 protein, and fragments of nucleic acid sequences encoding a biologically active ELMO1, ELMO2, ELMO3 or CED-12 protein. See FIGS. 1, 3, 5, 7, 9, 11 or 13. The terms “ELMO1 nucleic acid”, “ELMO2 nucleic acid”, “ELMO3 nucleic acid”, and “CED-12 nucleic acid” refer to the nucleic acid molecules that encodes the ELMO1, ELMO2, ELMO3 and CED-12 polypeptides, respectively. Fragments of the nucleic acid sequences described herein are useful as probes to detect the presence of the ELMO1, ELMO2, ELMO3 or CED-12 gene and mRNA in various species, or to screen cDNA libraries. The present invention also pertains to nucleic acid sequences encoding the ELMO1, ELMO2, ELMO3 or CED-12 protein, the fully complementary strands of these sequences and allelic variations thereof. Also encompassed by the present invention are nucleic acid sequences, DNA or RNA, which are substantially complementary to the DNA sequences encoding the ELMO1, ELMO2, ELMO3 or CED-12 protein, and which specifically hybridize with their DNA sequences under conditions of stringency known to those of skill in the art. Those conditions should be sufficient to identify DNA sequences with substantial sequence identity. As defined herein, substantially complementary means that the nucleic acid need not reflect the exact sequence of the ELMO1, ELMO2, ELMO3 or CED-12 DNA, but must be sufficiently similar in sequence to permit hybridization with ELMO1, ELMO2, ELMO3 or CED-12 DNA under stringent conditions. Conditions for stringency are described in e.g., Ausubel, F. M., et al., Current Protocols in Molecular Biology, (Current Protocol, 1994). For example, non-complementary bases can be interspersed in a nucleotide sequence, or the sequences can be longer or shorter than the ELMO1, ELMO2, ELMO3 or CED-12 DNA, provided that the sequence has a sufficient number of bases complementary to ELMO1, ELMO2, ELMO3 or CED-12 to allow hybridization therewith. Exemplary hybridization conditions are described herein and in standard texts, e.g., Ausubel, F. M., et al., Current Protocols in Molecular Biology, (Current Protocol, 1994). The ELMO1, ELMO2, ELMO3 or CED-12 DNA sequence, or a fragment thereof, can be used as a probe to isolate additional homologues. For example a cDNA or genomic DNA library from the appropriate organism can be screened with labeled DNA to identify homologues genes as described in e.g., Ausubel, F. M., et al., Current Protocols in Molecular Biology, (Current Protocol, 1994).

Stringency conditions for hybridization refers to conditions of temperature and buffer composition which permit hybridization of a first nucleic acid sequence to a second nucleic acid sequence, wherein the conditions determine the degree of identity between those sequences which hybridize to each other. Therefore, “high stringency conditions” are those conditions wherein only nucleic acid sequences that are very similar to each other will hybridize. The sequences can be less similar to each other if they hybridize under moderate stringency conditions. Still less similarity is needed for two sequences to hybridize under low stringency conditions. By varying the hybridization conditions from a stringency level at which no hybridization occurs, to a level at which hybridization is first observed, conditions can be determined at which a given sequence will hybridize to those sequences that are most similar to it. The precise conditions determining the stringency of a particular hybridization include not only the ionic strength, temperature, and the concentration of destabilizing agents such as formamide, but also on factors such as the length of the nucleic acid sequences, their base composition, the percent of mismatched base pairs between the two sequences, and the frequency of occurrence of subsets of the sequences (e.g., small stretches of repeats) within other non-identical sequences. Washing is the step in which conditions are set so as to determine a minimum level of similarity between the sequences hybridizing with each other. Generally, from the lowest temperature at which only homologous hybridization occurs, a 1% mismatch between two sequences results in a 1° C. decrease in the melting temperature (Tₘ) for any chosen SSC concentration. Generally, a doubling of the concentration of SSC results in an increase in the Tₘ of about 17° C. Using these guidelines, the washing temperature can be determined empirically, depending on the level of mismatch sought. Hybridization and wash conditions are explained in Current Protocols in Molecular Biology (Ausubel, F. M. et al., eds., John Wiley & Sons, Inc., 1995, with supplemental updates) on pages 2.10.1 to 2.10.16, and 6.3.1 to 6.3.6.

High stringency conditions can employ hybridization at either (1) 1×SSC (10×SSC=3 M NaCl, 0.3 M Na₃-citrate.2H₂O (88 g/liter), pH to 7.0 with 1 M HCl), 1% SDS (sodium dodecyl sulfate), 0.1-2 mg/ml denatured calf thymus DNA at 65° C., (2) 1×SSC, 50% formamide, 1% SDS, 0.1-2 mg/ml denatured calf thymus DNA at 42° C., (3) 1% bovine serum albumen (fraction V), 1 mM Na₂.EDTA, 0.5 M NaHPO₄ (pH 7.2) (1 M NaHPO₄=134 g Na₂HPO₄.7H₂O, 4 ml 85% H₃PO₄ per liter), 7% SDS, 0.1-2 mg/ml denatured calf thymus DNA at 65° C., (4) 50% formamide, 5×SSC, 0.02 M Tris-HCl (pH 7.6), 1×Denhardt's solution (100×=10 g Ficoll 400, 10 g polyvinylpyrrolidone, 10 g bovine serum albumin (fraction V), water to 500 ml), 10% dextran sulfate, 1% SDS, 0.1-2 mg/ml denatured calf thymus DNA at 42° C., (5) 5×SSC, 5×Denhardt's solution, 1% SDS, 100 μg/ml denatured calf thymus DNA at 65° C., or (6) 5×SSC, 5×Denhardt's solution, 50% formamide, 1% SDS, 100 μg/ml denatured calf thymus DNA at 42° C., with high stringency washes of either (1) 0.3-0.1×SSC, 0.1% SDS at 65° C., or (2) 1 mM Na₂EDTA, 40 mM NaHPO₄ (pH 7.2), 1% SDS at 65° C. The above conditions are intended to be used for DNA-DNA hybrids of 50 base pairs or longer. Where the hybrid is believed to be less than 18 base pairs in length, the hybridization and wash temperatures should be 5-10° C. below that of the calculated Tₘ of the hybrid, where Tₘ in ° C=(2×the number of A and T bases)+(4×the number of G and C bases). For hybrids believed to be about 18 to about 49 base pairs in length, the Tₘ in ° C.=(81.5° C.+16.6(log₁₀M)+0.41(% G+C)−0.61 (% formamide)−500/L), where “M” is the molarity of monovalent cations (e.g., Na⁺), and “L” is the length of the hybrid in base pairs.

Moderate stringency conditions can employ hybridization at either (1) 4×SSC, (10'SSC=3 M NaCl, 0.3 M Na₃-citrate.2H₂O (88 g/liter), pH to 7.0 with 1 M HCl), 1% SDS (sodium dodecyl sulfate), 0.1-2 mg/ml denatured calf thymus DNA at 65° C., (2) 4×SSC, 50% formamide, 1% SDS, 0.1-2 mg/ml denatured calf thymus DNA at 42° C., (3) 1% bovine serum albumen (fraction V), 1 mM Na₂.EDTA, 0.5 M NaHPO₄ (pH 7.2) (1 M NaHPO₄=134 g Na₂HPO₄.7H₂O, 4 ml 85% H₃PO₄ per litter), 7% SDS, 0.1-2 mg/ml denatured calf thymus DNA at 65° C., (4) 50% formamide, 5×SSC, 0.02 M Tris-HCl (pH 7.6), 1×Denhardt's solution (100 ×=10 g Ficoll 400, 10 g polyvinylpyrrolidone, 10 g bovine serum albumin (fraction V), water to 500 ml), 10% dextran sulfate, 1% SDS, 0.1-2 mg/ml denatured calf thymus DNA at 42° C., (5) 5×SSC, 5×Denhardt's solution, 1% SDS, 100 μg/ml denatured calf thymus DNA at 65° C., or (6) 5×SSC, 5×Denhardt's solution, 50% formamide, 1% SDS, 100 μg/ml denatured calf thymus DNA at 42° C., with moderate stringency washes of 1×SSC, 0.1% SDS at 65° C. The above conditions are intended to be used for DNA-DNA hybrids of 50 base pairs or longer. Where the hybrid is believed to be less than 18 base pairs in length, the hybridization and wash temperatures should be 5-10° C. below that of the calculated Tₘ of the hybrid, where Tₘ in ° C.=(2×the number of A and T bases)+(4×the number of G and C bases). For hybrids believed to be about 18 to about 49 base pairs in length, the Tₘ in ° C.=(81.5° C.+16.6(log₁₀M)+0.41(% G+C)−0.61 (% formamide)−500/L), where “M” is the molarity of monovalent cations (e.g., Na⁺), and “L” is the length of the hybrid in base pairs.

Low stringency conditions can employ hybridization at either (1) 4×SSC, (10×SSC=3 M NaCl, 0.3 M Na₃-citrate.2H₂O (88 g/liter), pH to 7.0 with 1 M HCl), 1% SDS (sodium dodecyl sulfate), 0.1-2 mg/ml denatured calf thymus DNA at 50° C., (2) 6×SSC, 50% formamide, 1% SDS, 0.1-2 mg/ml denatured calf thymus DNA at 40° C., (3) 1% bovine serum albumen (fraction V), 1 mM Na₂.EDTA, 0.5 M NaHPO₄ (pH 7.2) (1 M NaHPO₄=134 g Na₂HPO₄.7H₂O, 4 ml 85% H₃PO₄ per liter), 7% SDS, 0.1-2 mg/ml denatured calf thymus DNA at 50° C., (4) 50% formamide, 5×SSC, 0.02 M Tris-HCl (pH 7.6), 1×Denhardt's solution (100×=10 g Ficoll 400, 10 g polyvinylpyrrolidone, 10 g bovine serum albumin (fraction V), water to 500 ml), 10% dextran sulfate, 1% SDS, 0.1-2 mg/ml denatured calf thymus DNA at 40° C., (5) 5×SSC, 5×Denhardt's solution, 1% SDS, 100 μg/ml denatured calf thymus DNA at 50° C., or (6) 5×SSC, 5×Denhardt's solution, 50% formamide, 1% SDS, 100 μg/ml denatured calf thymus DNA at 40° C., with low stringency washes of either 2×SSC, 0.1% SDS at 50° C., or (2) 0.5% bovine serum albumin (fraction V), 1 mM Na₂EDTA, 40 mM NaHPO₄ (pH 7.2), 5% SDS. The above conditions are intended to be used for DNA-DNA hybrids of 50 base pairs or longer. Where the hybrid is believed to be less than 18 base pairs in length, the hybridization and wash temperatures should be 5-10° C. below that of the calculated Tₘ of the hybrid, where Tₘ in ° C.=(2×the number of A and T bases)+(4×the number of G and C bases). For hybrids believed to be about 18 to about 49 base pairs in length, the Tₘ in ° C.=(81.5° C.+16.6(log₁₀M)+0.41(% G+C)−0.61 (% formamide)−500/L), where “M” is the molarity of monovalent cations (e.g., Na⁺), and “L” is the length of the hybrid in base pairs.

Typically, the nucleic acid probe comprises a nucleic acid sequence (e.g., SEQ ID NO.:1) of sufficient length and complementarity to specifically hybridize to nucleic acid sequences which encode a ELMO1, ELMO2, ELMO3 or CED-12 protein. The requirements of sufficient length and complementarity can be easily determined by one of skill in the art. In fact, the Examples describe in detail such nucleic acid probes.

As used herein, an “isolated” gene or nucleotide sequence which is not flanked by nucleotide sequences which normally (e.g., in nature) flank the gene or nucleotide sequence (e.g., as in genomic sequences) and/or has been completely or partially purified from other transcribed sequences (e.g., as in a cDNA or RNA library). Thus, an isolated gene or nucleotide sequence can include a gene or nucleotide sequence which is synthesized chemically or by recombinant means. Nucleic acid constructs contained in a vector are included in the definition of “isolated” as used herein. Also, isolated nucleotide sequences include recombinant nucleic acid molecules and heterologous host cells, as well as partially or substantially or purified nucleic acid molecules in solution. In vivo and in vitro RNA transcripts of the present invention are also encompassed by “isolated” nucleotide sequences. Such isolated nucleotide sequences are useful for the manufacture of the encoded ELMO1, ELMO2, ELMO3 or CED-12 DNA, as probes for isolating homologues sequences (e.g., from other mammalian species or other organisms), for gene mapping (e.g., by in situ hybridization), or for detecting the presence (e.g., by Southern blot analysis) or expression (e.g., by Northern blot analysis) of related genes in cells or tissue.

As described above, the term “fragment” is meant to encompass a portion of the biologically active ELMO1, ELMO2, ELMO3 or CED-12 protein or polypeptide; or a nucleotide sequence described herein which is at least approximately 25 contiguous nucleotides to at least approximately 50 contiguous nucleotides or longer in length. Such fragments are useful as probes for diagnostic purposes, experimental tools, or in the case of nucleic acid fragments, as primers. A preferred embodiment includes primers and probes which selectively hybridize to the nucleic acid constructs encoding the ELMO1, ELMO2, ELMO3 or CED-12 protein. For example, nucleic acid fragments which encode any one of the domains described above are also implicated by the present invention.

Nucleic acid molecules can be inserted into a construct which can, optionally, replicate and/or integrate into a recombinant host cell, by known methods. The host cell can be a eukaryote or prokaryote and includes, for example, yeast (such as Pichia pastorius or Saccharomyces cerevisiae) bacteria (such as Escherichia coli or Bacillus subtilis), animal cells or tissue, insect Sf9 cells (such as baculoviruses infected SF9 cells) or a mammalian cells (somatic or embryonic cells, Chinese hamster ovary cells, HeLa cells, human 293 cells and monkey COS-7 cells).

The invention also provides vectors or plasmids containing one or more of each of the ELMO1, ELMO2, ELMO3 or CED-12 DNA. Suitable vectors for use in eukaryotic and prokaryotic cells are known in the art and are commercially available or readily prepared by a skilled artisan. Additional vectors can also be found, for example, in Ausubel, F. M., et al., Current Protocols in Molecular Biology, (Current Protocol, 1994) and Sambrook et al., “Molecular Cloning: A Laboratory Manual,” 2nd ED. (1989).

The nucleic acid molecule can be incorporated or inserted into the host cell by known methods. Examples of suitable methods of transfecting or transforming cells include calcium phosphate precipitation, electroporation, microinjection, infection, lipofection and direct uptake. “Transformation” or “transfection” as used herein refers to the acquisition of new or altered genetic features by incorporation of additional nucleic acids, e.g., DNA. “Expression” of the genetic information of a host cell is a term of art which refers to the directed transcription of DNA to generate RNA which is translated into a polypeptide. Methods for preparing such recombinant host cells and incorporating nucleic acids are described in more detail in Sambrook et al., “Molecular Cloning: A Laboratory Manual,” Second Edition (1989) and Ausubel, et al. “Current Protocols in Molecular Biology,” (1992), for example.

The host cell is then maintained under suitable conditions for expression and recovery of ELMO1, ELMO2, ELMO3 or CED-12 protein. Generally, the cells are maintained in a suitable buffer and/or growth medium or nutrient source for growth of the cells and expression of the gene product(s). The growth media are not critical to the invention, are generally known in the art and include sources of carbon, nitrogen and sulfur. Examples include Luria broth, Superbroth, Dulbecco's Modified Eagles Media (DMEM), RPMI-1640, M199 and Grace's insect media. The growth Media can contain a buffer, the selection of which is not critical to the invention. The pH of the buffered Media can be selected and is generally one tolerated by or optimal for growth for the host cell.

The host cell is maintained under a suitable temperature and atmosphere. Alternatively, the host cell is aerobic and the host cell is maintained under atmospheric conditions or other suitable conditions for growth. The temperature should also be selected so that the host cell tolerates the process and can be for example, between about 13°-40° C.

Methods for Assessment of ELMO1, ELMO2, ELMO3 or CED-12

The present invention includes methods of detecting the presence, absence or level of ELMO1, ELMO2, ELMO3 or CED-12, using standard immunochemistry methods. Methods that measure ELMO1, ELMO2, ELMO3 or CED-12 levels include several suitable assays. Suitable assays encompass immunological methods, such as FACS analysis, radioimmunoassay, flow cytometry, enzyme-linked immunosorbent assays (ELISA) and chemiluminescence assays. Any method known now or developed later can be used for measuring ELMO1, ELMO2, ELMO3 and CED-12 expression.

Antibodies can be raised to the ELMO1, ELMO2, ELMO3 and CED-12 polypeptides, analogs, and portions thereof, using techniques known to those of skill in the art. These antibodies can be polyclonal, monoclonal, chimeric, humanized or fragments thereof. The term “antibody” is intended to encompass polyclonal and monoclonal antibodies, and functional fragments thereof. The term “anti-ELMO1”, “anti-ELMO2”, “anti-ELMO3”, or “anti-CED-12” antibody includes monoclonal and/or polyclonal antibodies, and mixtures thereof. The terms polyclonal and monoclonal refer to the degree of homogeneity of an antibody preparation, and are not intended to be limited to particular methods of production. These antibodies can be used to purify or identify the ELMO1, ELMO2, ELMO3 and CED-12 polypeptides contained in a mixture of proteins, using techniques well known to those of skill in the art.

Anti-ELMO1, ELMO2, ELMO3 or CED-12 antibodies can be raised against appropriate immunogens, such as isolated and/or recombinant ELMO1, ELMO2, ELMO3 or CED-12 or portion thereof (including synthetic molecules, such as synthetic peptides). In one embodiment, antibodies are raised against an isolated and/or recombinant ELMO1, ELMO2, ELMO3 or CED-12 or portion thereof (e.g., a peptide) or against a host cell which expresses recombinant ELMO1, ELMO2, ELMO3 or CED-12. In addition, cells expressing recombinant ELMO1, ELMO2, ELMO3 or CED-12, such as transfected cells, can be used as immunogens or in a screen for antibody which binds receptor.

Any suitable technique can prepare the immunizing antigen and produce polyclonal or monoclonal antibodies. The art contains a variety of these methods (see e.g., Kohler et al., Nature, 256: 495-497 (1975) and Eur. J. Immunol. 6: 511-519 (1976); Milstein et al., Nature 266: 550-552 (1977); Koprowski et al., U.S. Pat. No. 4,172,124; Harlow, E. and D. Lane, 1988, Antibodies: A Laboratory Manual, (Cold Spring Harbor Laboratory: Cold Spring Harbor, N.Y.); Current Protocols In Molecular Biology, Vol.2 (Supplement 27, Summer ′94), Ausubel, F. M. et al., Eds., (John Wiley & Sons: New York, N.Y.), Chapter 11, (1991)). Generally, fusing a suitable immortal or myeloma cell line, such as SP2/0, with antibody producing cells can produce a hybridoma. Animals immunized with the antigen of interest provide the antibody producing cell, preferably cells from the spleen or lymph nodes. Selective culture conditions isolate antibody producing hybridoma cells while limiting dilution techniques produce them. Researchers can use suitable assays such as ELISA to select antibody producing cells with the desired specificity.

Other suitable methods can produce or isolate antibodies of the requisite specificity. Examples of other methods include selecting recombinant antibody from a library or relying upon immunization of transgenic animals such as mice.

According to the method, an assay can determine the level of ELMO1, ELMO2, ELMO3 or CED-12 in a biological sample. In determining the amounts of ELMO1, ELMO2, ELMO3 or CED-12, an assay includes combining the sample to be tested with an antibody having specificity for ELMO1, ELMO2, ELMO3 or CED-12, under conditions suitable for formation of a complex between antibody and ELMO1, ELMO2, ELMO3 or CED-12, and detecting or measuring (directly or indirectly) the formation of a complex. The sample can be obtained directly or indirectly, and can be prepared by a method suitable for the particular sample and assay format selected. The presence or absence of the ELMO1, ELMO2, ELMO3 or CED-12 polypeptide can be detected in an assay such as, for example, an ELISA or radioimmunoassay (RIA). The assay can be a direct detection or an indirect detection (e.g. a competitive assay).

For example, to determine the presence or absence of the ELMO1, ELMO2, ELMO3 and CED-12 polypeptides using an ELISA assay in a suitable sample, the method comprises:

(a) combining

(i) a suitable sample,

(ii) a composition comprising a murine anti-ELMO1, ELMO2, ELMO3 or CED-12 antibody as detector, such as (a) biotinylated MAb and HRP-streptavidin, or (b) HRP-conjugated Mab, and

(iii) a solid support, such as a microtiter plate, having an anti-ELMO1, ELMO2, ELMO3 or CED-12 capture antibody bound (directly or indirectly) thereto,

wherein the detector antibody binds to a different epitope from that recognized by the capture antibody, under conditions suitable for the formation of a complex between said anti-ELMO1, ELMO2, ELMO3 or CED-12 antibodies and ELMO1, ELMO2, ELMO3 or CED-12, respectively; and

(b) determining the formation of complex in said samples.

The presence of ELMO1, ELMO2, ELMO3 or CED-12 can also be determined in a radioimmunoassay. For example, the presence of ELMO1, ELMO2, ELMO3 or CED-12 can be assessed by an immunobinding assay comprising:

(a) obtaining a sample;

(b) contacting said sample with a composition comprising an anti-ELMO1, ELMO2, ELMO3 or CED-12 antibody, such as

(i) a murine anti-ELMO1, ELMO2, ELMO3 or CED-12 antibody comprising a radioactive label; or

(ii) a murine anti-ELMO1, ELMO2, ELMO3 or CED-12 antibody comprising a binding site for a second antibody which comprises a radioactive label,

preferably in an amount in excess of that required to bind the ELMO1, ELMO2, ELMO3 or CED-12, under conditions suitable for the formation of labeled complexes and

(c) determining (detecting or measuring) the formation of complex in said samples.

Suitable labels can be detected directly, such as radioactive, fluorescent or chemiluminescent labels. They can also be indirectly detected using labels such as enzyme labels and other antigenic or specific binding partners like biotin. Examples of such labels include fluorescent labels such as fluorescein, rhodamine, chemiluminescent labels such as luciferase, radioisotope labels such as ³²P, ¹²⁵I, ¹³¹I, enzyme labels such as horseradish peroxidase, and alkaline phosphatase, β-galactosidase, biotin, avidin, spin labels and the like. The detection of antibodies in a complex can also be done immunologically with a second antibody which is then detected (e.g., by means of a label). Conventional methods or other suitable methods can directly or indirectly label an antibody.

In performing the method, the levels of the ELMO1, ELMO2, ELMO3 or CED-12 that are distinct from the control. Increased levels of ELMO1, ELMO2, ELMO3 or CED-12 expression, as compared to a control, indicates increased levels of phagocytic activity, cell migration, or developmental processes. Similarly, decreased levels of ELMO1, ELMO2, ELMO3 or CED-12 expression, as compared to a control, indicates decreased levels of phagocytic activity, cell migration, or developmental processes. A control refers to a level of ELMO1, ELMO2, ELMO3 or CED-12 in a wild type organism, or from an organism not subjected to steps of the present invention (e.g., compounds, changes in temperature, light, etc.).

The ELMO1, ELMO2, ELMO3 or CED-12 expression can also be assayed by Northern blot analysis of mRNA from tissue samples or can be assayed by hybridization, e.g., by hybridizing one of the ELMO1, ELMO2, ELMO3 or CED-12 sequences provided herein (e.g., SEQ ID NO: 1, 3, 5, 7, 9, 11 or 13) or an oligonucleotide derived from one of the sequences, to a DNA-containing tissue sample. Such a hybridization sequence can have a detectable label, e.g., radioactive, fluorescent, etc., attached, to allow to detection of hybridization product. Methods for hybridization are well known, and described herein.

The cloning and functional characterization of C. elegans CED-12 and its homologues have permitted assay methods to be developed which allow identification of compounds which might inhibit or enhance ELMO1, ELMO2, ELMO3 or CED-12 activity or inhibit or enhance the transcription of these proteins. These can involve detection of the level of phagocytosis of dead cells or particles thereof, measurement of level of actin-cytoskeleton rearrangement or detection of the level of transcription of the ELMO1, ELMO2, ELMO3 or CED-12 proteins via a reporter gene such as GFP.

An assay for the identification of inhibitors and/or enhancers of phagocytosis can consist of a cell line stably or transiently transfected with ELMO 1, ELMO2, ELMO3 or CED-12. Cell lines can also be microinjected with purified protein or vectors expressing antisense RNA. The expression product can be a fusion protein with GFP. Non-transfected cells can be used in the assay also. The cell line can be a fibroblast cell line such as COSI, LR73, BHK 21, L929, CV1, Swiss 3T3, HT144, IMR32 or another fibroblast cell line. The cell line can also be an epithelial cell line such as HEPG2, MDCK, MCF7, 293, Hela, A549, SW48, G361, or any other epithelial cell line. The cell line can a primary line, such as human dermal FIBs, dermal keratinocytes, leucocytes, monocytes, macrophages, or any other primary cell line. Cells can be double transfected with other genes (such as lectin, CD14, SRA, CD36, ABC1, CED5, DOCK180) being from vertebrate (human, mouse) or invertebrate origin (C. elegans).

Phagocytosis assays consist of the addition of and uptake of particles and/or dead cells, by these cell lines. The particle can be fluorescently labeled, negatively charged latex beads of various sizes, opsonized heat or chemically killed bacteria and yeast in a variety of sizes, shapes. The cell can be a apoptotic neutrophils, apoptotic lymphocytes, apoptotic erythrocytes or any other apototic cell. These apoptotic cells can be opsonized and/or labeled with dyes or fluorescent dyes. The killed bacteria or yeast cells and the apoptotic cells are referred to as herein apoptotic particles.

Cells, transfected with ELMO1, ELMO2, ELMO3 or CED-12 or any other gene described herein, for example, nucleic acids of SEQ ID Nos: 1, 3, 5, 7, 9, 11 or 13, can be grown in a monolayer or in suspension. The apoptotic particles are added to the transfected cell. Phagocytosis can be followed by the uptake rate of the particles or dead cells. This can be measured by microscopy, by fluorescence microscopy, by quantitative spectrofluorometry and by flow cytometry. Cells and or particles can additionally be labeled with dyes, fluorescent dyes, antibodies and dyes of fluorescent dyes linked to antibodies prior to detection and measurement. Decrease or increase of the uptake of the apoptotic particles is a measurement for the influence of the transfected gene or genes in the phagocytosis.

Additionally, compounds can be also added to this assay to test their influence on the genes that are involved in the phagocytosis pathway. Transiently or stably transfected cells are grown in suspension or in a monolayer. A series of compounds is added to the cells prior to the addition of the apoptotic particles. The influence of the compounds can be measured by comparing the uptake rate of the apoptotic particles with and without the addition of the compound.

In another embodiment, cells are able to phagocytose apoptotic particles by engulfment of particles. This involves the reorganization of the actin cytoskeleton. Mammalian cells can be transiently or stably transfected with ELMO1, ELMO2, ELMO3 or CED-12, for example, with a nucleic acid have the sequence of nucleotides shown in any one of SEQ ID Nos: 1, 3, 5, 7, 9, 11 or 13. The genes can be expressed as a GFP fusion product. Cells can be double transfected, as described herein. The reorganization of the actin cytoskeleton can be visualized with fluorescent dyes linked to phalloidine, which interacts with F-actin. Reorganization of the cytoskeleton is an measurement for the engulfment induction by the transfected gene or genes. Transfected cells can be treated with particles or apoptotic cells as described herein. Reorganization of the cytoskeleton is visualized by microscopy or fluorescence microscopy. Also, compounds can be added to test their influence on the genes that are involved in the cytoskeleton reorganization related to the phagocytosis pathway and engulfment. The influence of the compounds can be measured by comparing the reorganization of actin cytoskeleton with and without the addition of the compound. Apoptotic particles can be added in this test to induce phagocytosis.

Another assay embodied by the present invention, non-transfected or transfected cell-lines such as those described above can be microinjected with purified ELMO1, ELMO2, ELMO3 or CED-12 protein, for example, a protein having the amino acid sequence as shown in SEQ ID Nos: 2, 4, 6, 8, 10, 12, 14, or 15. Microinjection can be done on the primary cell lines or the fibroblast cell lines or the other epithelial cells lines. The cell lines can be transfected with another gene prior to microinjections.

Transfected or non-transfected cell-lines as described above can also be microinjected with a vector expressing ELMO1, ELMO2, ELMO3 or CED-12 antisense RNA including antisense RNA in respect of any of the aforementioned proteins. Microinjection can be done on the primary cell lines or the fibroblast cell lines or the epithelial cell lines. The cell lines can be transfected with another gene prior to microinjection. Inhibitory effects of the antisense RNA by inhibition of the ELMO1, ELMO2, ELMO3 or CED-12 gene or genes involved in the ELMO1, ELMO2, ELMO3 or CED-12 pathway can be followed and detected as described herein. Compounds can be isolated which rescue the negative phenotype.

Phagocytosis assays to screen for ELMO1, ELMO2, ELMO3 or CED-12 inhibitor/enhancers in C.elegans are encompassed by the present invention. In particular, mutant worms lacking ELMO1, ELMO2, ELMO3 or CED-12 activity or with otherwise altered ELMO1, ELMO2, ELMO3 or CED-12 activity can be used. Alternatively, a transgenic worm transfected or transferred with ELMO1, ELMO2, ELMO3 or CED-12 DNA can be used. A series of compounds can be applied to the ELMO1, ELMO2, ELMO3 or CED-12 mutant worms or on worms harboring mutations in the ELMO1, ELMO2, ELMO3 or CED-12 pathway. Restoration of engulfment induced by the compounds can be visualized using Nomarski microscopy by counting cell corpses remaining in the head region of L1 larvae and in the gonads of the worms. A series of compounds can also be applied on humanized ELMO1, ELMO2 or ELMO3 mutant worms. Humanized worms are worms expressing the human ELMO1, ELMO2 or ELMO3 gene and are mutated for the C. elegans gene. Human ELMO1, ELMO2, ELMO3 rescues the mutant phenotype, as further described in the Examples. Compounds inhibiting or enhancing the ELMO1, ELMO2, ELMO3 or CED-12 phenotype can be selected by visualization of the engulfment phenotype using Nomarski microscopy and looking for cell corpses as described herein.

Hence, the present invention includes kits for the detection of ELMO1, ELMO2, ELMO3 or CED-12 or the quantification of ELMO1, ELMO2, ELMO3 or CED-12 having either antibodies specific for ELMO1, ELMO2, ELMO3 or CED-12 or a portion thereof, or a nucleic acid sequence that can hybridize to the nucleic acid of ELMO 1, ELMO2, ELMO3 or CED-12.

Therapeutic Uses of ELMO1, ELMO2, ELMO3 or CED-12

The process of cell death or apoptosis has been implicated in a wide range of diseases, including proliferative diseases (e.g., cancer), autoimmune diseases, various neurodegenerative diseases such as Amyotrophic Lateral Sclerosis, Huntington's Disease, and Alzheimer's Disease, stroke, myocardial heart infarct, and AIDS (Thompson, 1995). Thus a better understanding of the molecular events that underlie the removal of dying cells could lead to novel therapeutic interventions.

The process of recognition and engulfment of dying cells is extremely swift and efficient. In animals, it is essentially impossible to find a cell with apoptoic features that is not already within another cell. Such rapid recognition and phagocytosis of apoptotic cells is a crucial aspect of programmed cell death in vivo: unengulfed apoptotic bodies can undergo secondary necrosis, leading to inflammation. Failure to remove apoptotic bodies also exposes the body to novel epitopes (e.g., derived from caspase-generated protein fragments), possibly encouraging the development of autoimmune disease. Persistent apoptotic bodies can often be observed following chemotherapeutic intervention (which leads to extensive apoptosis) and are particularly abundant in solid tumors, in which clearance of cell corpses might be delayed.

It is likely that failure to properly dispose of apoptoic cells leads to human disease. Restoring proper phagocytosis is a valid therapy for certain types of inflammation and autoimmune diseases. Conversely, in some cases, cells that should be maintained are inappropriately recognized by the engulfment machinery and cleared from the body. Preventing the engulfment of such cells is also of great therapeutic value. Examples of such diseases might include neurodegenerative diseases and stroke, as well as sickle cell anemia.

In addition, activation of engulfment could be used for the same cases for which it is proposed to use activation of apoptosis, e.g., cancer. Indeed, specific activation within the cancer cells of the pro-engulfing signal would lead to the cells' removal and death without needing to activate the rest of the apoptotic machinery. This is particularly useful for highly resistant tumors in which crucial elements of the central apoptotic machinery have already been inactivated.

Accordingly, the present invention encompasses various therapeutic uses for ELMO1, ELMO2, ELMO3 and/or CED-12 proteins or nucleic acids. In particular, the present invention relates to treating or preventing diseases that involve a defect in ELMO1, ELMO2, or ELMO3, or a pathway thereof. An agonist of ELMO1, ELMO2, ELMO3 and/or CED-12 can be used to treat a disease in which the defect is a decrease in the expression of one or a combination of these proteins, or the defect results in decreased activity of the CrkII/DOCK180/Rac1 pathway. An antagonist of ELMO1, ELMO2, ELMO3 and/or CED-12 can be used to treat a disease that involves an increased expression (e.g., overexpression) of one or more of these proteins, or increased activity of the CrkII/DOCK180/Rac1 pathway. The present methods utilize various forms of antagonists.

An ELMO1, ELMO2, ELMO3 and/or CED-12 antagonist, as defined herein, means a compound that can inhibit, either partially or fully, phagocytosis of a dying cell or cell migration. Antagonists include antibodies, ribozymes, aptimers, or small molecule inhibitors and anti-sense molecules. An ELMO1, ELMO2, ELMO3 and/or CED-12 agonist refers to a compound that can enhance, either partially or fully, phagocytosis of a dying cell or cell migration.

In particular, a suitable antagonist that is an antisense molecule that can hybridize to the nucleic acid which encodes the target polypeptide (e.g.ELMO1, ELMO2, ELMO3 and/or CED). The hybridization inhibits transcription and/or synthesis of the protein. Antisense molecules can hybridize to all, or a portion of the nucleic acid. Producing such antisense molecules can be done using techniques well-known to those of skill in the art. For example, antisense molecules or constructs can be made using methods known in the art. DeMesmaeker, Alain, et al., Acc Chem. Res. 28:366-374 (1995), Setlow, Jane K., Genetic Engineering, 20:143-151 (1998); Dietz, U.S. Pat. No. 5,814,500, filed Oct. 31, 1996, entitled, “Delivery Construct for Antisense Nucleic Acids and Method of Use.”

An antagonists and agonist, as described herein, can also be used to treat proliferative diseases. Proliferative diseases are defined as a disorder/condition in which an abnormal rate of replication exists. An example of a proliferative disease is cancer. Various forms of the ELMO1, ELMO2, ELMO3 or CED-12 protein or nucleic acid, as described herein, can be administered and delivered to a mammalian cell (e.g., by virus or liposomes, or by any other suitable methods known in the art or later developed). The method of delivery can be modified to target certain cells, and in particular, cell surface receptor molecules or antigens present on tumor cells. Methods of targeting cells to deliver nucleic acid constructs are known in the art.

Thus, in accordance with another of its aspects the invention provides a method of treating, for example inflammation, autoimmune disease and cancer by administering to a patient an effective amount of a substance which enhances phagocytosis of apoptoic cells, in particular a substance which enhances the activity of ELMO1, ELMO2, ELMO3 or CED-12. Such substances include ELMO1, ELMO2, ELMO3 or CED-12 itself, a nucleic acid encoding ELMO1, ELMO2, ELMO3 or CED-12, an anti-sense nucleic acid to ELMO1, ELMO2, ELMO3 or CED-12 or compounds identified in any of the aforementioned assays as enhancers of ELMO1, ELMO2, ELMO3 or CED-12 or of transcription of ELMO 1, ELMO2, ELMO3 or CED-12.

In addition the invention also enables a method of treatment of, for example, neurodegenerative diseases, stroke and sickle-cell anaemia by administering to a patient an effective amount of a substance which inhibits phagocytosis of apoptotic cells, in particular a substance which inhibits the activity of ELMO1, ELMO2, ELMO3 or CED-12. Such substances include ELMO1, ELMO2, ELMO3 or CED-12 , a nucleic acid encoding ELMO1, ELMO2, ELMO3 or CED-12, an anti-sense nucleic acid to ELMO1, ELMO2, ELMO3 or CED-12 or compounds identified in any of the aforementioned assays as inhibitors of ELMO1, ELMO2, ELMO3 or CED-12 or of transcription thereof.

Pharmaceutical compositions comprising any of the above-mentioned therapeutic substances and a pharmaceutically acceptable carrier are also envisaged by the invention.

Mode and Manner of Administration

An ELMO1, ELMO2, ELMO3 and/or CED-12 protein or nucleic acid, agonist thereof or antagonist thereof can be administered using methods known in the art. For example, the mode of administration is preferably at the location of the target cells. As such, the administration can be nasally (as in administering a vector expressing ADA) or by injection (as in administering a vector expressing a suicide gene tumor). Other modes of administration (parenteral, mucosal, systemic, implant, intraperitoneal, etc.) are generally known in the art. The agents can, preferably, be administered in a pharmaceutically acceptable carrier, such as saline, sterile water, Ringer's solution, and isotonic sodium chloride solution. The entire protein or functional and/or biologically active portions thereof can be introduced into mammalian cells, as described above.

The terms “pharmaceutically acceptable carrier” or a “carrier” refer to any generally acceptable excipient or drug delivery device that is relatively inert and non-toxic. The compound can be administered with or without a carrier. An embodiment is to administer the compound (agonist or antagonist) directly to the target tissue (e.g., by injection), or, preferably, systemically (e.g., orally), but targeted to the particular tissue being treated. A compound can also be co-administered with other compounds known for treating the particular disease. Compounds that are co-administered with the compound of the present invention can be a known compound, or a compound that can be developed in the future. Exemplary carriers include calcium carbonate, sucrose, dextrose, mannose, albumin, starch, cellulose, silica gel, polyethylene glycol (PEG), dried skim milk, rice flour, magnesium stearate, and the like. Suitable formulations and additional carriers are described in Remington's Pharmaceutical Sciences, (17th Ed., Mack Pub. Co., Easton, Pa.).

Suitable carriers (e.g., pharmaceutical carriers) also include, but are not limited to sterile water, salt solutions (such as Ringer's solution), alcohols, polyethylene glycols, gelatin, carbohydrates such as lactose, amylose or starch, magnesium stearate, talc, silicic acid, viscous paraffin, fatty acid esters, hydroxymethylcellulose, polyvinyl pyrolidone, etc. Such preparations can be sterilized and, if desired, mixed with auxiliary agents, e.g., lubricants, preservatives, stabilizers, wetting agents, emulsifiers, salts for influencing osmotic pressure, buffers, coloring, and/or aromatic substances and the like which do not deleteriously react with the active compounds. They can also be combined where desired with other active substances, e.g., enzyme inhibitors, to reduce metabolic degradation. A carrier (e.g., a pharmaceutically acceptable carrier) is preferred, but not necessary to administer the compound.

For parenteral application, particularly suitable are injectable, sterile solutions, preferably oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-polyoxypropylene block polymers, and the like. Ampules are convenient unit dosages.

The actual effective amounts of compound or drug can vary according to the specific drug being utilized, the particular composition formulated, the mode of administration and the age, weight and condition of the patient, for example. As used herein, an effective amount of the drug is an amount of the drug which is capable of modulating phagocytic activity or cell migration. Dosages for a particular patient can be determined by one of ordinary skill in the art using conventional considerations, (e.g. by means of an appropriate, conventional pharmacological protocol).

A description of preferred embodiments of the invention follows.

### EXEMPLIFICATION

### EXAMPLE 1

### Experimental Procedures

Mutations and Strains

C. elegans were cultured as described by Brenner (1974). All strains were grown at 20° C. unless otherwise indicated. All mutant animals used in these studies were derived from the wild-type variety Bristol strain N2. Single nucleotide polymorphism mapping was performed with wild-type variety Hawaii, strain CB4856. The following mutations were described by Hodgkin, J., et al., Mapped genes and mutant phenotypes are listing in the Nematode Caenorhabditis elegans, W. B. Wood, ed. (Cold Spring Harbor, N.Y., Cold Spring Harbor Laboratory), pp. 502-559, except the CED-12 alleles, which were described in (Chung, S., et al., Nat Cell Biol 2, 931-937 (2000)) or in this study: LG I: dpy-5(e61), unc-29(e1072am), mec-8(e398am), CED-12(oz167), CED-12(bz187), CED-12(k145); CED-12(k149); CED-12(k156); CED-12(k158); lin-11(e566), unc-75(e950), CED-1(e1735). LG III: CED-6(n1813), ncl-1(e1865), CED-7(n1892). LG IV: CED-2(e1752), CED-5(n1812), CED-10(n1993), him-8(e1489), CED-3(n717). LG V: him-5(e1490).

Analysis and Quantification of Engulfment in C. elegans

The appearance and number of cell corpses were analyzed by mounting animals in a drop of M9 salt solution containing 30 mM NaN3 and observing the animals using Nomarski optics (Avery, L., et al., Cell 51, 1071-1078 (1987); Sulston, J. E., et al., Dev Biol 56, 110-156 (1977)). Animals were scored for cell corpses in the head just before hatching (three-fold stage) or just after hatching (four-cell gonad L1 stage; (Klass et al, Dev Biol 52, 1-18 (1976)). Germ cell corpses were assayed 24 to 36 hours after the L4 molt to adulthood. Average corpse numbers and the standard error of the mean (s.e.m.) were determined by the Statview II program (Abacus Concepts, Incorporated, Berkeley, Calif.).

Germ-Line Transformation and Genomic Rescue of CED-12

Transgenic animals were generated by using germline microinjection (Mello et al., EMBO J 10, 3959-3970 (1991)). YACS Y45G3, Y49H10, and Y39F7 were injected singly at 10 to 40 ng/μl into adult CED-12(oz167) hermaphrodites with the coinjection marker pRF4 at 80 ng/μl (Mello et al., 1991). pRF4 carries the mutated collagen gene rol-6(su1006gf) and confers a dominant roller (Rol) phenotype. Transgenic lines carrying stably transmitting extrachromosomal arrays were kept and assayed for persistent cell corpses under Nomarski optics. Rescue of CED-12(oz167) was considered to have occurred in lines where most late-stage embryos had four or fewer head cell corpses.

CED-12 Mapping with Single Nucleotide Polymorphisms (SNPs)

dpy-5(e61) unc-29(e1072am) CED-12(oz167) lin-11(e566) in an otherwise Bristol N2 background was crossed into wild-type Hawaii CB4856. 182 Dpy non-Lin and Lin non-Dpy recombinant F2 generation animals were picked and homozygous F3 individuals were isolated. F2 or F3 animals were scored for SNPs by PCR followed by restriction enzyme digest or sequencing. Some SNPs were found, while the others were identified by PCR amplification and sequencing of regions of interest from CB4856 genomic DNA. The true 5′ end of CED-12 was determined by reverse transcription-PCR from mixed stage him-5 RNA using the GibcoBRL 5′ RACE System for Rapid Amplification of cDNA ends, Version 2.0. The following primers were used: GSP1: ttccatgtaaaatctcc; GSP2: cttcaattagagcgggatccagaga; and primers specific to SL1 and SL2. Total mixed stage RNA was isolated as described by Burdine et al., Proc Natl Acad Sci USA 94, 2433-7 (1997).

Sequence Analysis

DNA fragments were sequenced at the core facilities at Cold Spring Harbor Laboratory facility or at the University of Virginia. Database searches were performed using the NCBI BLAST server. Sequence alignment was performed using the ClustalW and MultAlin algorithms (http://www.toulouse.inra.fr/multalin.html) and secondary structure predictions were performed using PredictProtein (http://www.embl-heidelberg.de/predictprotein). The motif searches were performed using Pfam, SMART and other protein analysis software that are publicly available on the WorldWideWeb (http://www.wi.mit.edu/bio, http://www.expasy.ch.tools, or http://www.ebi.ac.uk/Tools).

Northern Analysis

Total RNA was extracted from mixed-stage animals as described (Burdine et al., 1997), and polyA+ RNA was isolated from total RNA. The membrane for Northern blotting was prepared using the Ambion NorthemMax formaldehyde-based system and probed using standard radiolabeling techniques. The CED-12 probe was a 524bp EcoRI restriction fragment that included all of exon 3 and parts of exons 2 and 4. The loading control was a 1.8 kb PCR product of C. elegans eIF-4 (Baum et al., Neuron 19, 51-62 (1997)). A human 12-lane blot from Clontech, Inc. (Palo Alto, Calif.) containing 1 μg poly A+ RNA from 12 different human tissues was hybridized with a mouse ELMO1 probe (1.2kb fragment corresponding to nucleotides 983-2187) or an ELMO2 probe (748 bp fragment corresponding to nucleotides 1044-1792) and developed by autoradiography.

Transgenic Rescue Experiments

A full-length CED-12 cDNA was inserted into heat shock promoter vectors pPD49.78 and pPD49.83 that had been modified with Asc I and Fse I restriction sites for ease of shuttling inserts between vectors (Mello et al., Methods Cell Biol 48, 451-82 (1995). CED-12 was overexpressed before cell death occurs during embryonic development by allowing adult animals to lay embryos for about one hour, sealing the plates with parafilm and heat-shocking the plates in a 33° C. waterbath for 20-30 minutes. After one-hour recovery at 20° C., the adults were removed from the plates. Twelve to fourteen hours after heat shock, hatchling L1 larvae were scored for expression of the transgene and for corpses in the head region. CED-12 was also overexpressed after embryonic cell deaths occur by heat-shocking plates of embryos at all stages as described above. Five hours after the heat shock, hatchling L1 larvae (with four-celled gonads) were scored for expression of the transgenic marker and for corpses in the head region.

Cloning of ELMO1 AND ELMO2

Based on comparison of the mouse and human ESTs, two consensus primers (representing ELMO1 sequences) were designed and used in RT-PCR reactions of polyA+ RNA from RAW264.7 mouse macrophage line and in PCR amplification of a pACT human placental library and a human macrophage library. After confirmation of the amplified sequence as ELMO1, nested primers homologous to human ELMO1 and vector arms (of the pACT library) were designed and used in PCR reactions to obtain the 5′ end of ELMO1. The PCR products were cloned into pCR2.1 vector by TOPO cloning (Invitrogen). The appropriate colonies were identified by colony hybridization using ELMO1 internal oligonucleotides, and multiple clones were sequenced and compared to existing human ESTs and the human genome database. The sequence of human ELMO2, mouse ELMO1 and mouse ELMO2 were obtained by sequencing the EST clones AA216672, AI574349 and AA711524, respectively. The human EST AA216672, from which the hELMO-2 protein sequence was deduced, for unknown reasons contains an intron sequence between nucleotides 120-187 (numbered beginning with ATG). This was considered as an intron sequence based on computer analyses of other human ELMO2 ESTs that span this region, comparison to the human genome sequence and the mouse ELMO2 protein sequence. The complete coding sequence of the mouse ELMO1 and ELMO2 were PCR amplified from the EST clones and subcloned into the pEBB vector (Tanaka et al., Mol Cell Biol 15, 6829-37 (1995)), in which a C-terminal FLAG or a GFP tag was added. The coding sequence for enhanced green fluorescence protein (EGFP) was excised from pEGFP-NI (Clontech). The C-term version of ELMO1 (carrying deletion of amino acids 18-325) was generated after digesting the pEBB-ELMO1-FLAG plasmid with PflM I and BstB I enzymes, and addition of a linker to maintain the coding frame. The ELMO1-N-term (with deletion of amino acids 332-620) was generated after digestion of the pEBB-ELMO1-FLAG plasmid with BstB I and Nde I enzymes and addition of a linker to maintain the reading frame. Full-length Dock180, de1PS and de1GS mutant plasmids, Dock-CAAX, as well as the wt CrkII and CrkIIW169L plasmids were kindly provided by Michiyuki Matsuda (Japan) (Kiyokawa et al., 1998b).

In vitro Phagocytosis Assays

The phagocytic LR73 Chinese hamster ovary (CHO) cell line was cultured as previously described (Su et al., J Biol Chem 275, 9542-9 (2000)). Approximately 14-16 hours prior to transfection, 100,000-120,000 LR73 cells/well were seeded in a 24-well plate. Transfections of duplicate wells were performed using Lipofectamine 2000 reagent as per the manufacturer's recommendations (1.51 μl/reaction in serum-free medium) using ELMO1, CrkII, Rac1 or Dock180 plasmids at the indicated concentrations. When combinations of plasmids were used in transfection, carrier DNA (pEBB-FLAG vector alone) was added to keep the same plasmid concentration in the different samples. Twenty hours post-transfection, the cells were incubated with 2 μm or 0.1 μm carboxylate-modified red fluorescent beads (Sigma Chemical Co.) indicative of phagocytosis and pinocytosis, respectively, in serum-free medium for 2 hours. The wells were then aspirated and washed with cold PBS. The cells on the plate were trypsinized, resuspended in cold medium with 1% sodium azide, and 30,000 cells were analyzed (for each point) by two-color flow cytometry (Tosello-Trampont et al., J Biol Chem In Press. (2001). Forward scatter was used to gate out the unbound beads, which are much smaller in size than the LR73 cells. The untransfected cells were distinguished from the transfected cells through the GFP fluorescence due to the control GFP marker or ELMO1-GFP fluorescence. The untransfected cells in the same sample also provided an additional internal control in all experiments. The data were analyzed using Cell Quest software and the percentage of GFP positive cells that engulfed the “red” beads was analyzed by first gating on the GFP population. Controls for binding versus internalization suggest that the majority of the fluorescence measured in the assay is due to internalization rather than binding (except for the first peak in the histogram (Tosello-Trampont et al., J Biol Chem In Press. (2001). It is noteworthy that the engulfment assays were performed at 20 hours post-transfection, when the ELMO1 transfected cells appeared morphologically indistinguishable from GFP alone expressing cells.

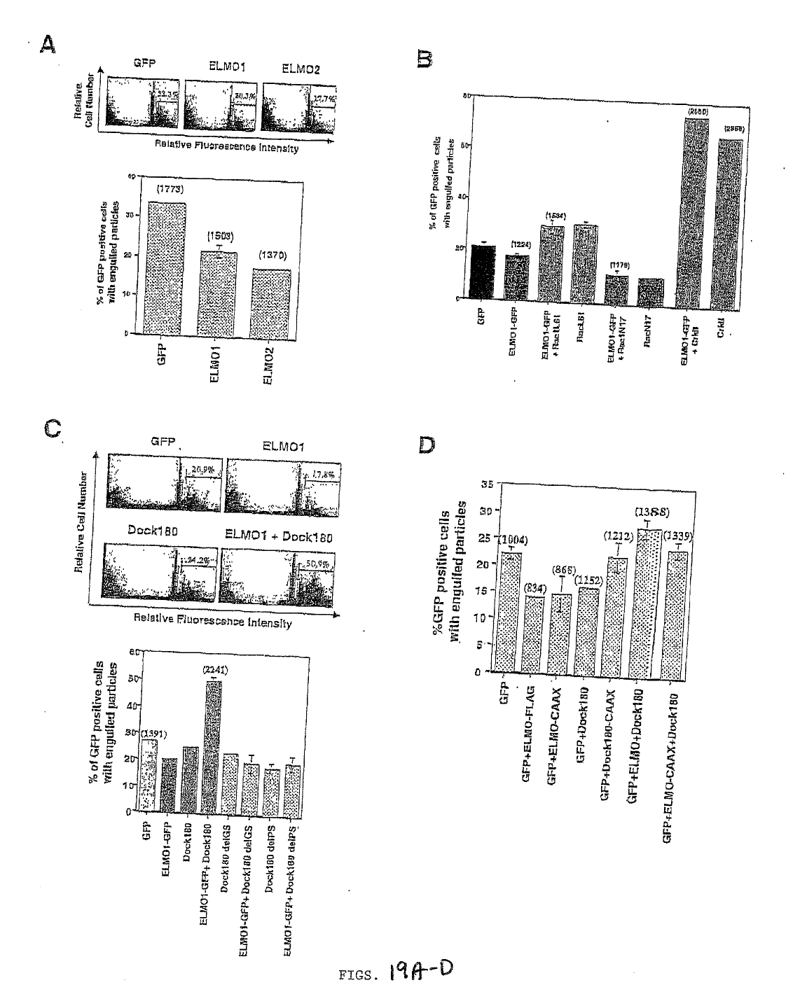

To obtain the data shown in FIG. 19, LR73 cells were transiently transfected in duplicate wells of a 24-well plate with plasmids coding for GFP, ELMO1-GFP or ELMO2-GFP. 20 hours post-transfection, the cells were incubated with “red” labeled 2 μm carboxylate-modified beads, and 30,000 cells per sample were analyzed by two-color flow cytometry. The fraction of GFP positive cells that had taken up the red beads along with the standard deviations of the two duplicate samples is shown. The histogram profiles of the “green” cells for their mean fluorescence intensity (MFI), indicative of the “efficiency” of bead uptake, are also shown. The sharp first peak in the gate setting was excluded, as it reflects fluorescence due to bound rather than engulfed particles. Effect of coexpression of Rac1L61 or CrkII with ELMO1. LR73 cells were transiently transfected as above with plasmids encoding GFP or ELMO1-GFP (0.8 μg) together with Rac1L61, Rac1N17 or CrkII (0.7 μg). Carrier DNA (FLAG-tagged vector alone) was added to keep the concentration of plasmid used the same in the different samples. Twenty hours after transfection, the engulfment assay was performed. Functional cooperation between ELMO1 and Dock180. ELMO1-GFP plasmid (0.8 μg) was cotransfected with Dock180 (1.8 μg), de1GS Dock180 (1.3 μg) or de1PSDock180 (1.7 μg) (plasmid concentrations were altered to normalize for the differences in plasmid size). Carrier DNA (FLAG-tagged vector alone) was added to keep the concentration of plasmid used as the same in the different samples. The phagocytosis assay was performed as described herein. Other plasmid concentrations, done as part of dose response in the same experiment, gave qualitatively similar results. ELMO1-CAAX does not show a gain of function. LR73 cells were transiently transfected with plasmids encoding ELMO1-FLAG, ELMO1-CAAX, His-Dock180, Dock180-CAAX or the different combinations, together with a GFP marker plasmid. The phagocytosis by GFP positive cells was then analyzed as above.

Immunoprecipitations and Immunoblotting

COS-7 or 293T cells were transiently transfected with 3 μg of ELMO1-FLAG or other plasmids (except 10 μg of Dock180 plasmids, which were less well expressed). 36 hours post-transfection, the cells were harvested and lysed in 1% Nonidet P-40 and immunoprecipitated using anti-FLAG antibody directly coupled to sepharose (clone M2) (Sigma Chemical Co.) or with the indicated antibodies and Protein A+G sepharose (Santa Cruz Biotechnology Inc., Santa Cruz, Calif.). The immunoblotting was performed using the following antibodies: anti-Dock180 (N-19 and C-19, Santa Cruz Biotechnology), anti-FLAG (clone M2, Sigma Chemical Co.), anti-CrkII (Transduction Laboratories, Ky.), anti-His (Invitrogen) and anti-GFP (Santa Cruz Biotechnology).

Detection of Protein Complex Formation Using the Yeast Two-hybrid System

GAL4 DNA binding (GBD) and activation domain (GAD) fusion constructs were co-transformed into the yeast reporter strain HF7c (R1). Transformants were plated on synthetic medium lacking leucine and tryptophan (DO-Leu-Trp). Individual colonies were patched on DO-Leu-Trp plates and subsequently replica plated onto DO-Leu-Trp-His plates. After 4 days, growth was evaluated. As controls, GBD-CED-5 and GAD-CED-12 were cotransfected with pGAD and pGBT empty vectors respectively. No growth on DO-Leu-Trp-His was observed. The GBD-Rac constructs were obtained by cloning BamH1/Sal1 fragments from LBD-RacWT, RacV12 and RacN17 (Joneson et al., 1996) into the corresponding sites in pGBT11 (Van Aelst, 1997). GBD-CED-5 was constructed by cloning the full-length cDNA coding for CED-5 into pAS2 vector (gift from Nina Cromheecke), whereas pGAD-CED-12 was constructed by cloning cDNA encoding full-length CED-12 into pGADstop vector.

Confocal Microscopy

LR73 cells were transfected with the indicated plasmids using Lipofectamine as described above. Twenty-four hours post-transfection, cells were washed twice with cold PBS/0.%BSA, fixed for 10 minutes with PBS/3% paraformaldehyde at room temperature (RT), and then washed twice with cold PBS/0.1%BSA. After permeabilization for 10 min with PBS/0.1%BSA/0.1%Triton X-100 at RT, the cells were equilibrated by incubation with PBS/2%BSA for 10 min at RT, and incubated with phalloidin-rhodamine (Molecular Probes, Eugene, Oreg.) (5 μl/200 μl PBS/1%BSA) for 30 min at RT in the dark, and washed 4 times and analyzed using an Olympus Confocal microscope.

Membrane localization of ELMO 1-GFP

The indicated plasmids were transfected into LR73 cells using Lipofectamine 2000 reagent at the following concentrations: GFP alone (0.4 μg), ELMO1-GFP (0.6 μg), GFP/Dock180 (0.4/1.0 μg), ELMO1-GFP/Dock180 (0.6/1.0 μg), ELMO1-GFP/Dock180/CrkII (0.6/1.0/0.4 μg) and ELMO1-GFP/Dock180/CrkII/Rac1N17 (0.6/1.0/0.4/1.5 μg). 24-hours after transfection, the cells were stained with phalloidin-rhodamine and analyzed by confocal microscopy. The regions of overlay of the green ELMO1-GFP fluorescence with the red fluorescence of phalloidin-rhodamine are represented as yellow. The images shown are representative of multiple cells with similar phenotype on the same slide and are representative of two independent experiments.

### EXAMPLE 2

### Results

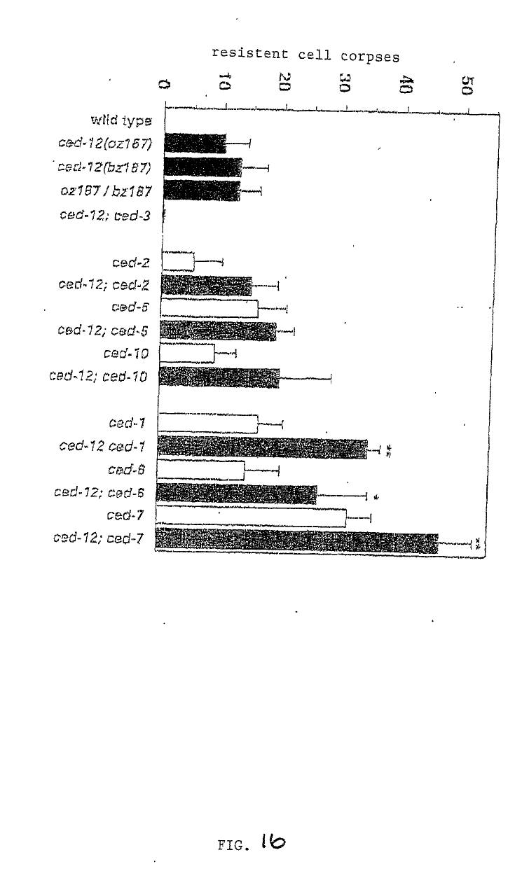

All six CED-12 alleles, described herein, share a similar set of defects, which include a pronounced deficiency in the removal of apoptotic cell corpses, migrational and morphological defects in the hermaphrodite gonad, and a partially penetrant embryonic lethality (Tables 1, 2; FIG. 16). These defects are described in more detail below.

TABLE 1
Mutations in ced-12 cause defects in engulfment of cell corpses, DTC migration and embryonic
development.

| | | | |
|---|---|---|---|
| | | | oz167; |
| wild type | ced-12(oz167) | ced-12(bz187) | ced-3 (n717) |

| | | | | |
|---|---|---|---|---|
| Phenotype | n | n | n | n |

Engulfment defects

| | | | | | | | | |
|---|---|---|---|---|---|---|---|---|
| Cell corpses in 3-fold embryo head | 0.06 | 100 | 22 | 5 | ND | | 0 | 60 |
| Cell corpses in L1 head | 0 | 100 | 7.3 | 64 | 12.8 | 12 | 0.1 | 100 |
| Cell corpses in 24-hr adult germ line | 1.3 | 10 | 6.1 | 25 | ND | | 0 | 30 |
| DTC migration defects | | | | | | | | |
| Aggregated DTC migration defects (%) | 0 | 286 | 63 | 102 | 39 | 103 | 53 | 195 |
| Other defects | | | | | | | | |
| Embryonic death or malformation (%) | <0.1 | 514 | 23 | 97 | 17 | 139 | 24 | 125 |
| Vulval malformations (%) | 0 | 143 | 2 | 1415 | 1 | 2580 | 2 | 775 |
| Sterility (%) | 0 | 14 | 17 | 12 | 8 | 12 | ND | |
| Average brood size | 290 | 9 | 172 | 15 | 163 | 12 | ND | |

Analysis of ced-12 mutant alleles oz167 and bz187 revealed a number of developmental defects. Mutations in the caspase gene ced-3, which prevent apoptotic cell death, only suppressed the engulfment defect in the oz167 background, indicating that the other defects are independent of apoptosis. ND, not determined. n, number of animals analyzed, except for brood size, where n represents the number of broods scored.

TABLE 2
ced-12/elmo promotes distal tip cell migration

A. ced-12 is required for multiple aspects of gonad development in hermaphrodites

| | Hermaphrodite | | | | | | |
|---|---|---|---|---|---|---|---|
| | anterior gonad defects (%) | | | posterior gonad defects (%) | | | Male |
| Genotype | extra turn | branching | other | extra turn | branching | other | defects (%) |
| ced-12(bz187) | 9 | 4 | 5 | 44 | 13 | 8 | 0 |
| ced-12(k145) | 15 | 0 | 3 | 31 | 7 | 8 | 1 |
| ced-12(k149) | 13 | 3 | 6 | 34 | 23 | 8 | 1 |
| ced-12(k156) | 18 | 0 | 6 | 26 | 8 | 4 | 1 |
| ced-12(k158) | 54 | 0 | 11 | 44 | 2 | 16 | 0 |
| ced-12(oz167) | 29 | 0 | 8 | 42 | 0 | 8 | 4 |

B. elmo expression can partially rescue DTC migration defect in ced-12 mutants

| | | posterior | |
|---|---|---|---|
| | anterior | gonad | |
| | gonad | defects | |
| Genotype | defects (%) | (%) | n |
| ced-12(oz167) | 29 | 42 | 100 |
| ced-12(oz167); opEx478[sur-5::GFP] | 16 | 43 | 81 |
| ced-12(oz167); opEx458[eft-3::ced-12] | 4 | 9 | 92 |
| ced-12(oz167); opEx481[eft-3:: elmo1-FLAG] | 6 | 15 | 83 |
| ced-12(oz167); opEx475[eft-3:: elmo2-FLAG] | 5 | 16 | 81 |

A, Percentages of misshapen hermaphrodite and male gonads in late L4 or young adult animals grown at 20° C. were examined. Males used were of him-5(e1490) background except for bz187 males, which were scored in a wild-type background. him-5(e1490) males had a 1% DTC migration defect. n = 160 for k145, k149, k156 and k158 hermaphrodites and 100 for bz187 and oz167 hermaphrodites and all males. Extra turn: extra-turning on dorsal muscle;
branching: branching occured at or after second turn, and resulted in an extra small ‘appendage”; other: all other visilble defects, e.g. meandering or premature stop on dorsal muscle. B, Extra turn defect in anterior and posterior gonad of animals transgenic for ced-12 or tagged elmo-expressing cDNAs. All extrachromosomal arrays are marked with sur-5::GFP. n, number of animals analyzed.

CED-12 is Required for the Removal of Dying Cells

Programmed cell death is a common feature during C. elegans embryonic development: 113 of the 671 cells generated during embryogenesis are subsequently eliminated by an apoptotic process. Normally, corpses of cells that die during embryogenesis are engulfed within an hour, and all of the dying cells are removed before the animal hatches. In contrast, in worms carrying the CED-12(lf) mutant allele, many cell corpses persisted into the first larval stage after hatching and beyond (Table 1). Wild-type and CED-12(oz167) late embryos (3-fold stage) were observed. By this stage of development, all embryonic cell deaths have been cleared in the wild-type embryo, whereas the CED-12 embryo contains many persistent cell corpses.

Apoptotic cell death also occurs in the adult germ line, where it acts as a homeostatic mechanism to regulate the number of syncytial nuclei that are allowed to differentiate into oocytes. In CED-12(lf) mutant animals, dying germ cells were not swiftly engulfed and accumulated in the germline, such that over four times the normal number of germ cell corpses were seen in CED-12(oz167) animals of the same age (Table 1). The persistent cell corpse phenotype of CED-12 mutants was abrogated if cell death was prevented (Table 1), e.g., through inactivation of the CED-3 caspase homologue (if no cells die, then no cell corpses are formed). In addition to its role in the engulfment of apoptotic corpses, CED-12 is also required for the efficient removal of cells that die by necrotic-like processes, suggesting that its function is not restricted to the apoptotic program.

CED-12 is Required for Embryonic Development

Indeed, further analysis revealed that CED-12 is required for a large number of developmental processes. Most dramatically, approximately one fifth of the CED-12(oz167) mutant embryos suffered from abnormal development, usually with lethal consequences (Table 1). The range of defects was broad, with some embryos failing to initiate proper morphogenesis and others aborting at various stages during the elongation process. The lethality to a single defect could not be unambiguously assigned. However, the wide range of terminal phenotypes would be consistent with a general, but low penetrance failure in cell migrations.

Mutations in CED-12 also cause sublethal developmental defects. Some embryos were small but were otherwise morphologically normal; this can be due to formation of small oocytes in gonad arms with enlarged diameter. In addition, larval development was frequently delayed: in CED-12(bz187) mutant animals synchronized at hatching, over 10% of all progeny were at least one larval molt (greater than twelve hours) behind their siblings by the time of the final molt, while none of the wild-type progeny were developmentally retarded. Finally, CED-12 mutants showed reduced fertility: CED-12 mutants generated barely half as many progeny (brood size) as wild-type animals, with a significant fraction of worms being completely sterile (Table 1), possibly the result of extreme forms of the gonadogenesis defects described below.

CED-12 is Required for DTC Migration and Gonadal Morphogenesis

In wild-type nematodes, the distal tip cells guide the developing hermaphrodite gonad into a bibbed U-shaped form. Distal tip cell (DTC) migration defects were observed and Normarski pictures were taken. In wild-type hermaphrodites, the DTC follows a stereotyped migration pattern resulting by the late L4 stage in a U-shaped gonad. In over half of CED-12(1f) hermaphrodites, the migration of at least one of the two distal tip cells was misdirected, resulting in gonads with abnormal morphology (Tables 1 and 2). Variable migration defects in CED-12 mutants result in gonad arms that often fail to reflex, or make extra turns. CED-12 mutants also occasionally show a distended proximal tube as well as short branches or distensions. In addition, gonad arms frequently had an uneven diameter, with a very distended proximal end and/or small branches or extrusions at bends (Table 2A). These defects can explain why some CED-12(1f) hermaphrodites were sterile or had a low brood size. Male gonads consist of a single arm that also migrates extensively. However, loss of CED-12 function only weakly affected migration defects in males, if at all (Table 2A). The reason for this sexual bias in the requirement for CED-12 in gonadal migration is unknown, although a similar bias has also been observed for other genes that affect DTC migration. Despite their normal gonad morphology, CED-12 males have difficulty in siring progeny, suggesting that CED-12 is required for at least one other aspect of male mating. The embryonic lethality and the diverse cell migration defects were still present in CED-12(1f); CED-3(1f) double mutants (Table 1), confirming that CED-12 acts in these processes independently of its role in engulfment.

CED-12 Acts in the CED-2/CED-5/CED-10 Pathway

Genetic studies suggest that the engulfment CED genes act in two partially redundant, parallel pathways with CED-1, CED-6, and CED-7 acting in one pathway, and CED-2, CED-5, and CED-10 in the other. To determine the relationship of CED-12 to the two pathways, double mutants between CED-12 and all other engulfment CED genes were constructed and the persistent apoptotic cell corpse numbers in single and double mutants were compared (FIG. 16). Double mutants harboring CED-12 and CED-1, CED-6, or CED-7 exhibited significantly increased numbers of apoptotic corpses compared to the single mutants, consistent with a model in which CED-12 is involved in a different process from this first group. In contrast, a much more modest increase in the number of unengulfed corpses in CED-12; CED-2 and CED-12; CED-10 double mutants were observed, while the CED-12; CED-5 double mutants exhibited persistent corpse counts similar to the CED-5 single mutant. The weak corpse enhancement for double mutants with CED-2 and CED-10 could have resulted from the use of non-null alleles in the assay. Overall, the data suggest that CED-12 acts in the same genetic pathway as CED-2, CED-5, and CED-10. Consistent with this categorization, DTC migration defects were only observed in CED-2, CED-5, CED-10, and CED-12 mutants, but not in CED-1, CED-6, or CED-7 mutants (Table 1), suggesting that only the former group is required for this process.

Cloning of CED-12

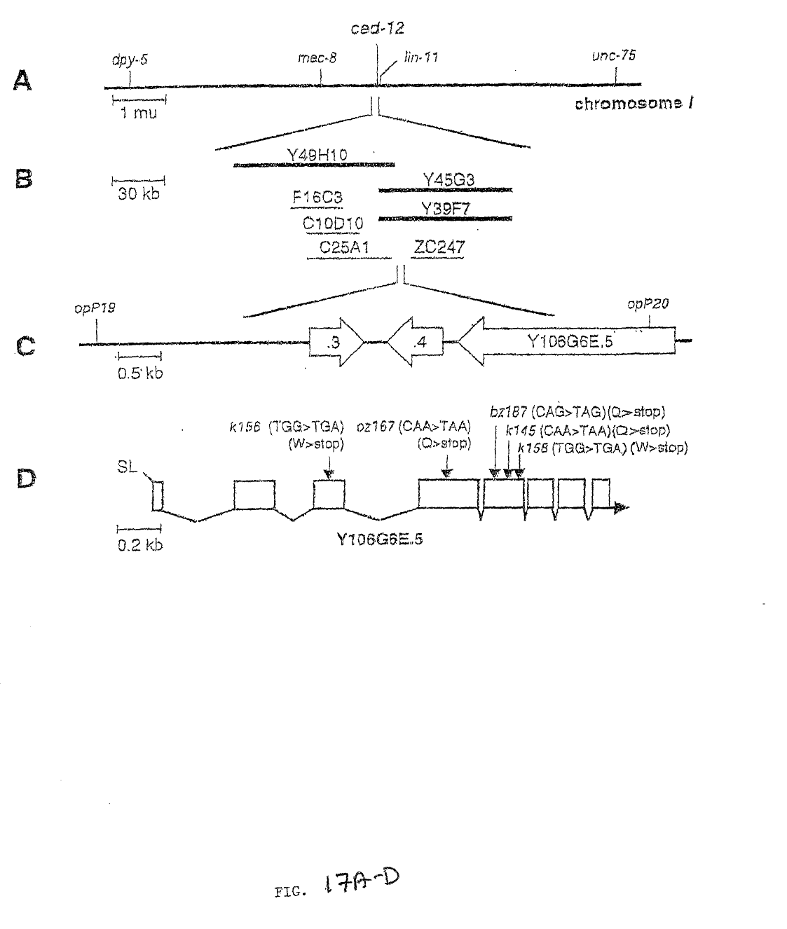

CED-12 had previously been mapped to chromosome I close to and to the left of lin-11. CED-12(oz167) animals with the yeast artificial chromosomes Y45G3, Y49H10, and Y39F7 were rescued, after the attempts to rescue with cosmids spanning this region were unsuccessful (FIG. 17B, see below). Using a set of single nucleotide polymorphisms (SNPs) (See Example 1), CED-12 was further restricted to one of three genes in the cosmid gap covered by Y106G6E (FIG. 17C). Two lines of evidence led us to conclude that CED-12 corresponds to Y106G6E.5. First, mutations in Y106G6E.5 in five of the six CED-12 mutant strains were identified (FIG. 17D, FIG. 17A). All mutations introduced nonsense codons, resulting in premature termination of translation. Second, expression of a full-length Y106G6E.5 cDNA fully rescued the engulfment and DTC migration defect of CED-12 mutants (Table 2B).

The intron-exon structure of CED-12 was determined by sequencing six independent cDNAs and performing reverse transcription polymerase chain reaction (RT-PCR) amplification on CED-12(+) mRNA. The CED-12 mRNA is trans-spliced to either SL1 or SL2 splice leaders. There were no splice variations among the six sequenced cDNAs. Furthermore, northern blot analysis of polyA+ mRNA of wild-type and two CED-12 mutant animals showed a single band, slightly larger than the predicted size based on the cDNAs (2.4 vs. 2.2 kb), most likely due to the presence of a polyA tail in the mature message. Northern blot analysis reveals a single 2.5 kb CED-12 transcript. The levels of CED-12 mRNA are reduced in CED-12 mutants, but relatively normal in other cell death mutant backgrounds (except for the CED-3 mutant, the reason for which is unclear). A probe recognizing the eIF-4 initiation factor transcript was used to control for loading. Compared to the wild-type, CED-12 message levels appeared reduced in CED-12(oz167) and CED-12(bz187) mutants, likely because of nonsense-mediated decay of these message by the smg system.

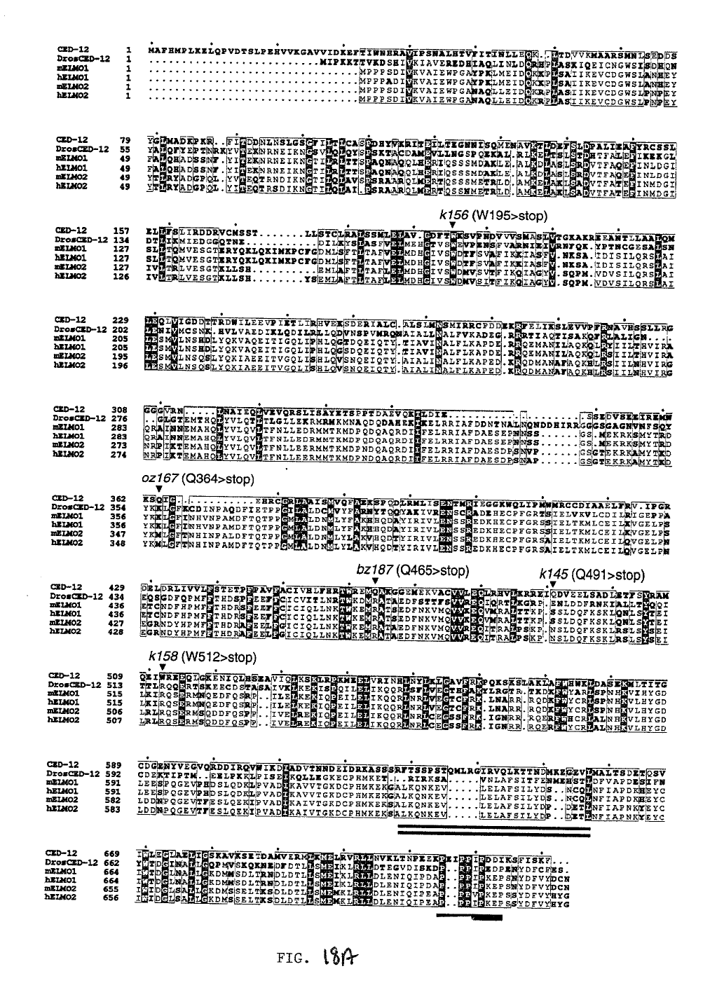

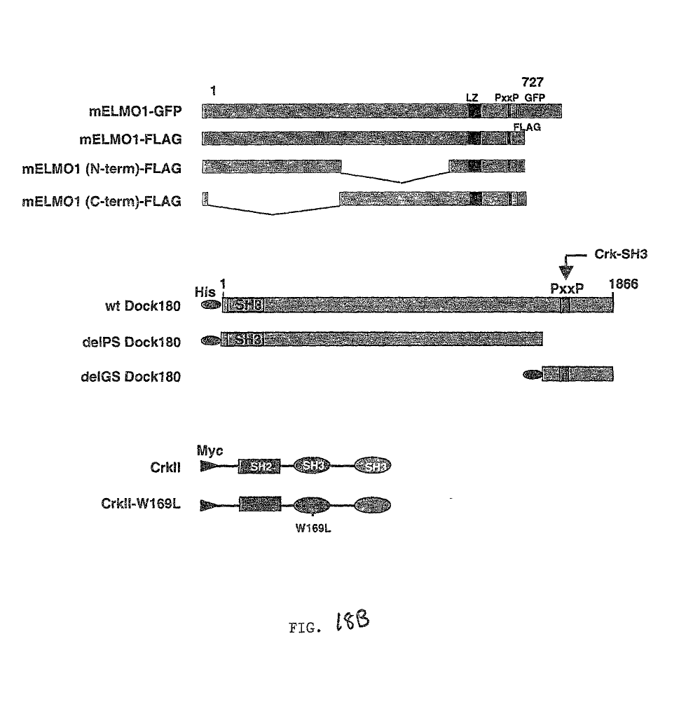

CED-12 encodes a novel protein of 731 amino acids (FIG. 18A) and its molecular function is not obvious from the sequence analysis. However, CED-12 is clearly conserved through evolution, possessing at least one homologue in Drosophila (previously uncharacterized gene CG5336), and two in mice and humans (see below, FIG. 18A).

CED-12 Promotes the Engulfment of Apoptotic Cells and is Required in the Engulfing Cell

To conclusively demonstrate that Y106G6E.5 corresponds to CED-12, it was determined whether the full-length cDNA could rescue the engulfment defect in CED-12(1f) animals. CED-12(1f) worms that were transgenic for a fall length Y106G6E.5 cDNA under the control of the C. elegans heat shock promoters hsp-16.2 and hsp-16.48 were generated (See Example 1). These two promoters together drive expression in almost all somatic cells upon heat shock stimulus, including cells fated to die and engulfing cells. These transgenic animals were tested for rescue by exposing early embryos to a pulse of heat just before the first wave of developmental cell deaths. These animals were then scored shortly after hatching. Overexpression of Y106GE.5 cDNA almost completely eliminated the persistence of cell corpses in the heads of young transgenic CED-12(1f) larvae (Table 3A).

TABLE 3
Overexpression of a ced-12 cDNA rescues ced-12 mutants, but not other engulfment mutants

| | | | | |
|---|---|---|---|---|
| Genotype | Transgenic | Heat Shock | Cell Corpses in L1 Head | n |

A. Overexpression of a ced-12 cDNA rescues the ced-12 engulfment defect

| | | | | |
|---|---|---|---|---|
| ced-12(oz167) | N/A | − | 7.3 ± 1.0 | 64 |
| ced-12(oz167) | N/A | + | 8.0 ± 1.6 | 42 |
| ced-12(oz167); opEx434[hs::ced-12] | − | − | 5.7 ± 1.0 | 42 |
| ced-12(oz167); opEx434[hs::ced-12] | + | − | 2.7 ± 1.1 | 11 |
| ced-12(oz167); opEx434[hs::ced-12] | − | + | 7.2 ± 1.6 | 36 |
| ced-12(oz167); opEx434[hs::ced-12] | + | + | 0.3 ± 0.3 | 21 |
| ced-12(oz167); opEx435[hs::ced-12] | − | − | 7.3 ± 2.2 | 16 |
| ced-12(oz167); opEx435[hs::ced-12] | + | − | 3.2 ± 0.9 | 22 |
| ced-12(oz167); opEx435[hs::ced-12] | − | + | 9.1 ± 2.4 | 20 |
| ced-12(oz167); opEx435[hs::ced-12] | + | + | 0.1 ± 0.1 | 27 |
| ced-12(oz167); opEx431[hs::gfp] | − | + | 13.3 ± 1.9 | 8 |
| ced-12(oz167); opEx431[hs::gfp] | + | + | 10.9 ± 1.0 | 20 |

B. Overexpresion of ced-12 promotes the engulfment of persistent cell corpses

| | | | | |
|---|---|---|---|---|
| ced-12(oz167); opEx434[hs::ced-12] | − | + | 12.9 ± 0.8 | 19 |
| ced-12(oz167); opEx434[hs::ced-12] | + | + | 1.3 ± 0.4 | 15 |

C. hsp::ced-12(+) does not rescue other engulfment mutant animals

| ced-1(e1735); opEx435[hs::ced-12] | − | + | 14.8 ± 1.6 | 11 |
|---|---|---|---|---|
| ced-1(e1735); opEx435[hs::ced-12] | + | + | 16.8 ± 0.7 | 32 |
| ced-6(n1813); opEx434[hs::ced-12] | − | + | 8.4 ± 1.1 | 12 |
| ced-6(n1813); opEx434[hs::ced-12] | + | + | 10.0 ± 0.8 | 34 |
| ced-7(n1892); opEx435[hs::ced-12] | − | + | 21.3 ± 2.1 | 12 |
| ced-7(n1892); opEx435[hs::ced-12] | + | + | 19.0 ± 1.3 | 12 |
| ced-2(e1752); opEx434[hs::ced-12] | − | + | 10.5 ± 1.0 | 12 |
| ced-2(e1752); opEx434[hs::ced-12] | + | + | 13.6 ± 1.1 | 28 |
| ced-5(n1812); opEx435[hs::ced-12] | − | + | 22.5 ± 1.6 | 13 |
| ced-5(n1812); opEx435[hs::ced-12] | + | + | 21.6 ± 2.6 | 11 |
| ced-10(n1993); opEx435[hs::ced-12] | − | + | 9.6 ± 1.2 | 13 |
| ced-10(n1993); opEx435[hs::ced-12] | + | + | 9.0 ± 0.9 | 20 |
| Transgenic and non-trangenic progeny from transgenic parents were given a heat shock pulse, and the number of persistent cell corpses in the head of freshly hatched L1 larvae were scored. Data shown are average ± s.e.m. Two independent transgenic extrachromosomal arrays (opEx434 and opEx435) expressing a full-length ced-12 cDNA under the control of the heat-shock promoters hsp16-41 and hsp-16.2 were tested. As a negative control, a transgenic array (opEx431) expressing GFP under the | | | | |
| #same promoter combination was used. n, number of animals scored. N/A, not applicable. Note that, in section A, transgenic animals without heat-shock also show a partial rescue, likely due to leakiness of the promoter in the arrays tested. | | | | |

To determine whether CED-12 acts in the dying or the engulfing cells, heat shocked embryos were tested five hours before hatching, by which time most embryonic cell deaths had already occurred. Previous work has shown that the hsp-16.2 and hsp-16.48 heat shock promoters cannot direct new gene expression in late apoptotic corpses. Rescue, therefore, can only occur if the expression of CED-12 in living, engulfing cells was sufficient for phagocytosis of cell corpses. The number of persistent cell corpses was dramatically reduced when CED-12 expression was induced after embryonic cell death (Table 3B). This result indicates that CED-12 expression in the engulfing cell is sufficient for engulfment.

CED-12 Acts Upstream of CED-10

It was determined whether overexpressed CED-12 could compensate for the loss of engulfment potential in the other engulfment genes. This method has previously been used to show that CED-6 acts downstream of CED-1 and CED-7, and that CED-10 acts downstream of CED-2 and CED-5. The hs::CED-12 transgene was crossed into the background of mutations in other engulfment genes and tested whether induction of CED-12(+) expression could restore phagocytosis. Overexpression of CED-12(+) had no significant rescue of any of the other mutants tested, suggesting that CED-12 acts either independently, upstream, or at the same step as the other engulfment genes (Table 3C).

Cloning of Mammalian Orthologues of CED-12

To clone the mammalian orthologue of CED-12, the DNA and protein databases were searched using the C. elegans and Drosophila CED-12 sequence, and identified multiple human and murine expressed sequence tags (ESTs) with homology to CED-12. By designing primers based on some of the EST sequences, PCR products from a human macrophage library and from RNA of the murine macrophage cell line RAW264.7 line were obtained. The full-length human CED-12 gene was cloned from the library using a PCR strategy (See Example 1). This human CED-12 gene was predicted to encode a 727 amino acid polypeptide with 44% similarity to C. elegans CED-12 and 65% similarity to the Drosophila CED-12 homologue. Comparing this sequence with the human genome sequence database revealed that in addition to the CED-12 gene that was isolated from the library (located on chromosome 7), there was also a second homologous gene located on chromosome 20. A human EST clone (AA216672) that corresponded to this second CED-12 gene provided the sequence of a 722aa polypeptide. Two murine ESTs (AI574349 and AA711524), which corresponded to the two human CED-12 genes, provided the sequence of the mouse CED-12 genes. The mammalian CED-12 orthologues were named ELMO (genes involved in engulfment and cell motility), and refer to the products of the two CED-12 genes as ELMO1 and ELMO2. The identified sequences appeared to encode the full-length open reading frame for both ELMO1 and ELMO2 genes of human and mouse based on the following criteria: i) Consensus Kozak sequences exist upstream of the predicted first ATG; ii) Comparison of several human and mouse ESTs to each other and with the existing genome database information indicated stop codons in all three reading frames upstream of the predicted first ATG; iii) At the 3′ end, in frame STOP codons were identified in all cases and in a human ELMO1 EST (KIAA0281), the polyadenylation signal could also be identified; iv) The predicted N- and C-termini of the mammalian proteins align well with the predicted N- and C-termini of the Drosophila and worm proteins.

The mouse and human ELMO1 polypeptides are highly conserved with 98% amino acid identity, as is also the case with ELMO2 from the two species. When ELMO1 was compared to ELMO2, there was 75% identity and 88% similarity in both species. In comparison, both ELMO1 and ELMO2 have about 44% similarity to the C. elegans CED-12 and 65% similarity to the Drosophila CED-12 (FIG. 18A). Genome gazing suggests that C. elegans and Drosophila contain only one gene representing CED-12, while there are two family members in humans. The two mammalian gene products have greater similarity to each other than to either the fly or the worm CED-12 protein, suggesting that a duplication of the CED-12 gene occurred later in evolution.

A northern blot of polyA+ RNA from various tissues using an ELMO1 probe showed that it is widely expressed in all tissues as a 4.2 kb transcript, although at lower levels in colon and small intestine compared to other tissues. In brain, another transcript of 2.2 kb was also noted. Northern analysis of polyA+ RNA from various tissues using an ELMO1 or ELMO2 probe (See Example 1) demonstrated that both genes are widely expressed. ELMO2 had a slightly different expression pattern, with greater expression in skeletal muscle and kidney, and less expression in the spleen. The greater than 100 ESTs that exist in the database for human and murine ELMO1 and ELMO2 originate from a large variety of tissues, confirming that both genes are widely expressed. RT-PCR using primers specific for ELMO1 or ELMO2 showed expression of both genes in the macrophage line RAW264.7, murine B cell line A20, and Jurkat T cell line, as well as in the LR73 cell line used in the subsequent experiments described below.

The predicted CED-12 and ELMO proteins appear unique with no significant similarity to other proteins in the public databases, and with no obvious catalytic domain. Computer analysis (See Example 1) predicted the ELMO1 and ELMO2 to be soluble cytoplasmic proteins. Other than a potential SH3 binding PxxP motif near the C-terminus (conserved from worm to human), and a putative leucine zipper around amino acid 630 (that was found in all of the mammalian proteins, but not in the worm or fly proteins), no other significant domains or motifs were identified.

Mammalian ELMO can Partially Substitute for CED-12 to Promote DTC Migration

Previous work on cell death genes has shown that orthologous genes often are functionally conserved between nematodes and mammals, performing similar roles in their respective organisms. To test whether CED-12/ELMO might also be functionally conserved, transgenic animals expressing tagged ELMO1 and ELMO2 clones under the control of the ubiquitous promoter eft-3 were generated, and the ability of these transgenes to rescue the engulfment and DTC migration defects of CED-12 mutants was tested. While the ELMO constructs did not significantly rescue the engulfment defect, constitutive expression of ELMO1 or ELMO2 could almost completely rescue the DTC migration defect (Table 2B), strongly suggesting that ELMO1 and ELMO2 can interact with the appropriate CED-12 partners in C. elegans. It is unknown why ELMO did not rescue the engulfment defect of CED-12 mutants. A similar partial rescue of CED-5 mutants by the CED-5 homologue Dock180 using a different promoter has been reported, suggesting that DTC migration might be generally easier to rescue than engulfment of apoptotic corpses.

ELMO1 Cooperates with CrkII and Dock180 in Engulfment

To determine whether ELMO1 or ELMO2 plays a role in phagocytosis in mammalian cells, FLAG or GFP-tagged constructs of mouse ELMO1 and ELMO2 were generated (See FIG. 3C for a schematic representation of the constructs used). The phagocytic fibroblast line LR73 was transiently transfected with ELMO1-GFP, ELMO2-GFP or as a control, GFP alone (in duplicate). Twenty hours post-transfection, the population of cells was incubated with “red” fluorescent 2 μm carboxylate-modified latex beads, which is shown to serve as a simplified target that mimics the negatively charged apoptotic cells in experiments designed to test CrkII and Rac function in engulfment (Tosello-Trampont et al., 2001). Using a flow cytometry-based engulfment assay, the fraction of GFP expressing cells that had engulfed the negatively charged red beads by two-color analysis (See Example 1) was determined. Surprisingly, expression of ELMO1 or ELMO2 resulted in a reduced uptake, compared to GFP alone (FIG. 19A). Such a reduction in uptake was seen in multiple (>20) independent experiments. The flow cytometry-based engulfment assay also allowed us to monitor the mean fluorescence intensity (MFI) of individual cells, roughly indicative of the number of beads taken up, and gave an indication of their “efficiency of uptake”. Often, the ELMO-GFP expressing cells that did engulf the particles were less efficient, as the MFI of ELMO-GFP positive cells was lower than that of control cells expressing GFP alone (FIG. 19A). However, in some experiments, a significant decrease in MFI values was not observed, while a reduced uptake due to ELMO expression was seen.

While the precise reason for the inhibition is not known, overexpression of ELMO1 or ELMO2 could have sequestered or interfered with other proteins involved in engulfment (see below). Expression of unrelated proteins such as the adapter protein Shc or glutathione S-transferase (GST) did not cause an inhibition of phagocytosis. The inhibition due to ELMO1-GFP expression was not an artifact of GFP tagging as the expression of FLAG-tagged ELMO also gave similar results (FIG. 19D). The uptake of 0.1 μm carboxylate-modified beads (indicative of pinocytosis) was unaffected by ELMO1-GFP expression, arguing against a general inhibitory effect of ELMO on plasma membrane events.

Based on the genetic studies in C. elegans described herein, in mammalian cells, ELMO1 also functions in the CrkII/Dock180/Rac pathway during engulfment. Coexpression of any of these proteins with ELMO was tested to determine whether it would “rescue” the inhibition of engulfment. Since CED-10/Rac is considered the most downstream protein in the pathway, an activated form of Rac (Rac1L61) was tested to determine whether it can overcome the inhibition due to ELMO1. Expression of Rac1L61 enhanced the uptake compared to GFP alone expressing cells and the cotransfection of ELMO1 did not cause an inhibition of the Rac1L61 mediated uptake (FIG. 19B). The dominant negative Rac1N17 mutant led to an inhibition of uptake, which was unaffected by coexpression of ELMO1. These data are consistent with a model where CED-10/Rac functions downstream of CED-12/ELMO.

The effect of CrkII expression was tested in conjunction with ELMO1 (FIG. 19B). Transfection of CrkII alone led to an enhancement of engulfment, similar to what was observed previously (Tosello-Trampont et al., 2001). Cotransfection of CrkII and ELMO1 led to a further enhancement of phagocytosis, compared to transfection with CrkII alone (FIG. 19B). Moreover, the “efficiency” of uptake after CrkII and ELMO1-GFP cotransfection was greater than transfection with CrkII alone. The small but reproducible increase in uptake, as well as the greater MFI due to coexpression of ELMO1 with CrkII was consistently seen in multiple experiments. These data were suggestive of a functional cooperation between ELMO1 and CrkII.

The effect of overexpression of CED-5/Dock180 was tested either alone or with ELMO1-GFP. Transient transfection of a plasmid encoding Dock180 alone also led to a modest inhibition of uptake, similar to what was previously observed with ELMO (FIG. 19C). This small decrease in uptake was consistently seen in multiple experiments. In contrast, cotransfection of Dock180 and ELMO1-GFP led to a nearly two-fold increase in bead uptake compared to the GFP-alone control. The “efficiency” of bead uptake by the Dock180+ELMO1 transfected cells was also increased (FIG. 19C). These data suggest a functional cooperation between Dock180 and ELMO during engulfment. They also indirectly supported the hypothesis that the inhibition of engulfment observed when transfected with ELMO1 alone might have been due to sequestration of Dock180.

To determine which regions of Dock180 are required for cooperation with ELMO, either the de1PS mutant of Dock180, which lacks the CrkII binding sites, or the de1GS mutant, which lacks most of the N-terminus of the protein but is still capable of binding to CrkII was expressed (see schematic in FIG. 3C). Neither deletion construct cooperated with ELMO1 (FIG. 19C); thus, both regions of Dock180 are necessary for enhancement of engulfment following ELMO1/Dock180 coexpression.

It has been previously demonstrated that while cytoplasmic overexpression of Dock180 had little effect on the actin cytoskeleton, membrane-targeting of Dock180 through a CAAX farmesylation sequence at its C-terminus led to cell shape changes mediated through Rac 1. To test if membrane targeting of ELMO1 might also provide a gain of function, LR73 cells were co-trasnfected with an ELMO1-CAAX construct and a GFP marker plasmid. ELMO1-CAAX showed an inhibition of uptake similar to that observed with ELMO1-FLAG (FIG. 19D). In contrast, the Dock180-CAAX transfection led to a better “efficiency” of uptake than GFP alone transfection, although the percentage of transfected cells engulfing particles was not enhanced. These data suggest that forced membrane targeting of ELMO1 per se does not provide a gain of ELMO1 function.

Biochemical Interaction Between ELMO and Dock180

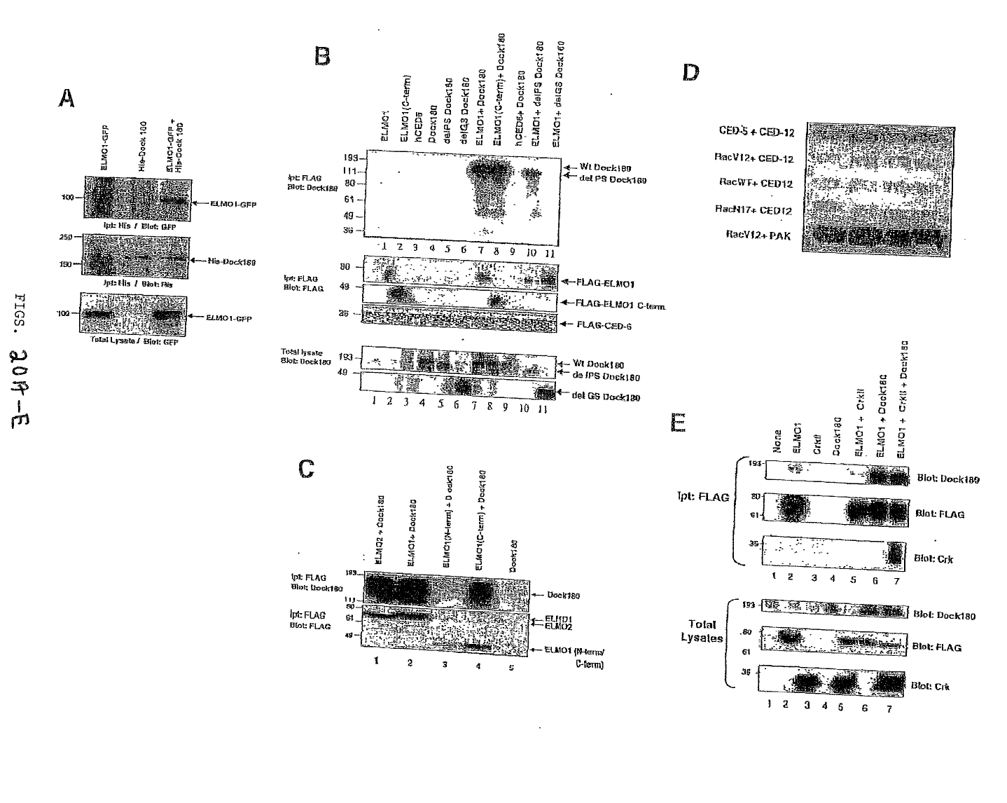

It was determined whether there might be a physical interaction between ELMO1 and Dock180, Rac or CrkII. After transient transfection of COS-7 cells with plasmids encoding ELMO1-GFP and His-Dock180, ELMO-GFP was specifically coprecipitated with Dock180 (FIG. 20A). Similarly, FLAG-tagged ELMO1 specifically coprecipitated His-Dock180 only when both proteins were coexpressed (FIG. 20B, lane 7). In a control immunoprecipitation, Dock180 was not coprecipitated by FLAG-tagged human CED-6 (which is also involved in engulfment and contains a leucine zipper) (Su et al., 2000) (lane 9). When the de1PS or deIGS mutants of Dock180 was co-expressed with ELMO1, the de1PS mutant bound to ELMO1 (FIG. 20B, lane 10), but the de1GS mutant failed to interact with ELMO1 (FIG. 20B, lane 11). Since the de1PS mutant lacks the proline-rich motifs necessary for CrkII binding to Dock180, it appears that ELMO binding can occur in the absence of the CrkII-Dock180 interaction.

Crude analysis of the region of ELMO required for Dock180 binding suggested that the C-terminal construct of ELMO1 (See Schematic in FIG. 3C) was necessary and sufficient for interaction with Dock180 (FIG. 20B, lane 8, and FIG. 20C, lane 4), while the N-terminal construct did not coprecipitate Dock180 (FIG. 20C, lane 3). ELMO2 also coprecipitated Dock180 (FIG. 20C, lane 1), indicating that both forms of ELMO can interact with Dock180. It was tested whether the C. elegans CED-12 protein can interact with mammalian Dock180; however, these experiments were inconclusive as the worm protein was poorly expressed in COS-7 or 293T cells. When the interaction of the worm CED 12 and the worm CED 5 in a yeast two-hybrid assay were examined, an interaction between these two proteins was detected, indicating that the CED-12/ELMO and CED-5/Dock180 interaction is evolutionarily conserved (FIG. 20D). In the same two-hybrid assay, an interaction between wt or mutant forms of Rac and CED-12 wass not detected (FIG. 20D).

Whether ELMO1 can interact with CrkII was also tested. After overexpression of CrkII with FLAG-ELMO1 in 293T cells, CrkII was not detected in ELMO1 immunoprecipitates (FIG. 20E, lane 5). Under the same conditions, Dock180 was readily coprecipitated with ELMO1 (FIG. 20E, lane 6). Moreover, Crk immunoprecipitates also failed to coprecipitate ELMO1. Since Dock180 can interact with both ELMO1 and CrkII, it was determined whether a complex of ELMO1/Dock180/CrkII could be formed. Indeed, cotransfection of all three genes resulted in the coprecipitation of CrkII with ELMO1 (FIG. 20E, lane 7). The trimeric complex was not observed when the de1PS or de1GS mutants of Dock180 were coexpressed. These data suggested that ELMO1, Dock180 and CrkII can form a trimeric complex, most likely through Dock180 bridging ELMO1 and CrkII.

Functional Cooperation Between CrkII/Dock180/ELMO Proteins

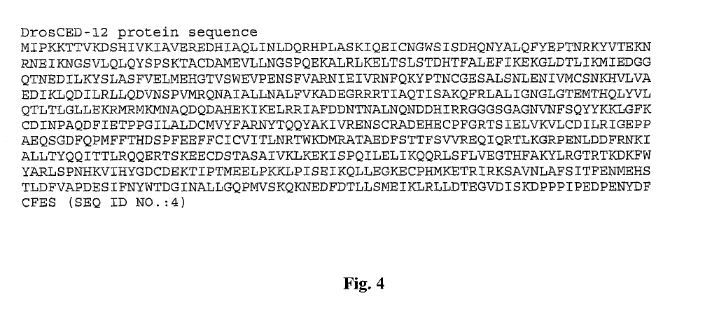

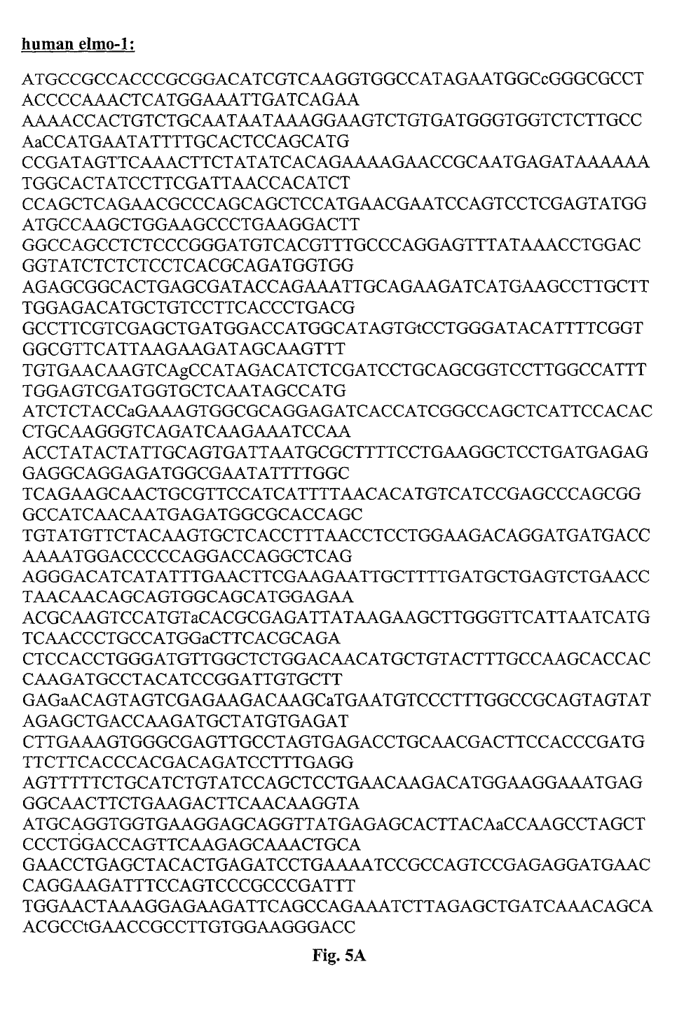

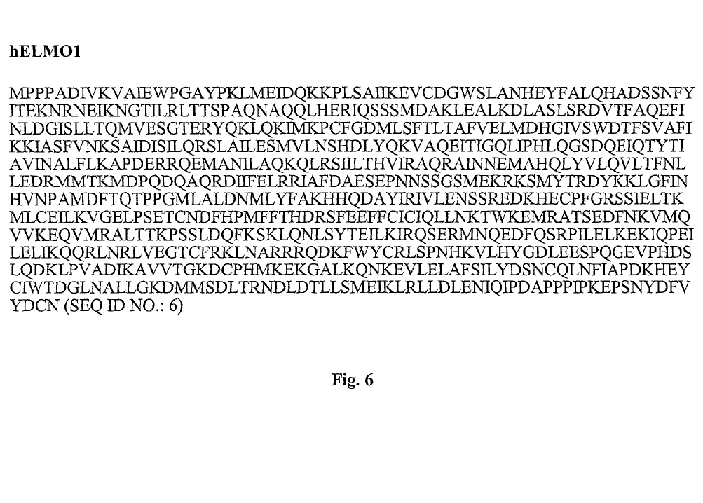

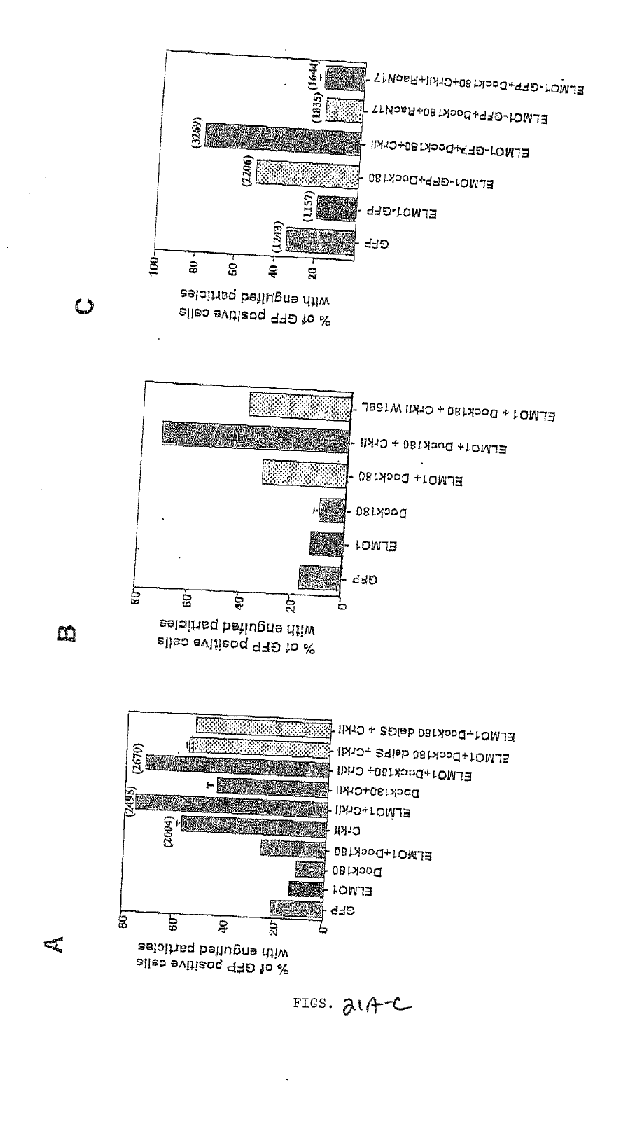

To determine whether the trimeric complex between ELMO1/Dock180/CrkII might be functionally relevant during phagocytosis, LR73 cells transiently transfected with plasmids coding for ELMO1-GFP alone or with either CrkII or Dock180, or the three proteins combined. As noticed previously (FIG. 4B and 4C), an enhanced uptake was seen when ELMO1 was cotransfected with either Dock180 or CrkII. When CrkII, Dock180 and ELMO1 were cotransfected together, the efficiency of bead uptake (as determined by MFI) was the highest among all conditions tested (seen in multiple experiments), although the triple transfection did not lead to a greater percentage of particle uptake compared to ELMO1+CrkII coexpression (FIG. 21A). Simultaneous expression of all three proteins was required to obtain optimal bead uptake, since cotransfection of CrkII and Dock180, without ELMO1, led to a lower uptake than overexpressing CrkII alone (FIG. 21A). Moreover, mutants of Dock180 that failed to show a biochemical interaction with either ELMO1 or CrkII, or a mutant of CrkII that fails to interact with Dock180, did not show the synergy in the triple transfections (FIGS. 6A and 6B). Taken together, these data suggest that the biochemical interaction observed between ELMO1/Dock180/CrklI plays a functional role during phagocytosis.

It was determined whether the enhanced uptake due to cotransfection of ELMO1 with CrkII and Dock180 is mediated through CED-10/Rac. Rac1N17, a dominant negative form of Rac1, inhibited the enhanced uptake due to ELMO1+Dock180 as well as ELMO1+Dock180+CrkII cotransfection (FIG. 21C). The enhancement due to CrkII+ELMO1 transfection was also inhibited by Rac1N17. All of the transfected proteins were expressed as determined by immunoblotting of total cell lysates from parallel samples. Taken together, these data suggested that ELMO1, CrkII and Dock180 function upstream of Rac during phagocytosis, consistent with the proposed order of gene function in C. elegans.

ELMO1 Induces Cell Shape Changes and Localizes to Membrane Ruffles

Rac is essential for actin polymerization at the leading edges of the cell and is thought to facilitate cell migration through the protrusion of lamellipodia. Since CED-12 is required for cell migration in the worm and ELMO1 appeared to function upstream of Rac1 in engulfment and was capable of rescuing the DTC migration defect in CED-12 deficient worms, it was determined whether ELMO1 can promote actin polymerization and alter cell shape. LR73 cells were transfected with the indicated plasmids and analyzed the localization of ELMO1 through its GFP fluorescence and the actin polymerization through rhodamine-labeled phalloidin. LR73 cells normally have a spindle-like shape; this shape was not affected by expression of GFP or Dock180. In contrast, expression of ELMO1-GFP, which localized in the cytoplasm when transfected alone, caused about two thirds of the cells to adopt a polygonal shape (based on counting 100-200 cells from multiple fields). Cells transfected with Dock180 and ELMO1-GFP were quite similar in shape to ELMO1-GFP alone expressing cells; however, a minor, but detectable, colocalization of ELMO1-GFP was observed with F-actin in these doubly transfected cells and some localization of ELMO1-GFP to the plasma membrane. However, a striking change in cell shape, along with relocalization of ELMO1-GFP to abundant membrane ruffles, was observed seen when CrkII was cotransfected with ELMO1 and Dock180. When CrkII alone was coexpressed with ELMO1, there was ruffling of the membrane, although it was less pronounced, and there was also some localization of ELMO1 to the membrane that was detectable. Since ruffling has been shown to be a Rac1-dependent event (Ridley et al., 1992), the effect of coexpressing dominant negative Rac1 along with ELMO1/CrkII/Dock180 was tested. The ruffling due to expression of ELMO1/Dock180/CrkII was lost or significantly diminished when Rac1N17 was cotransfected. It is noteworthy that despite the overall diminished staining for F-actin due to Rac1N17 coexpression, the localization of ELMO1-GFP to the membranes (seen in multiple cells in the field) could still be detected. This is consistent with a model that ELMO1 functions upstream of Rac1, in which case Rac1N17 would not be expected to affect ELMO1 localization per se. Taken together, these data suggest that ELMO1 can localize to membrane ruffles through a process influenced by Dock180 and CrkII proteins, and in turn, could regulate the cytoskeleton in a Rac1-dependent manner.

All references, patents, and patent applications cited herein are hereby incorporated by reference in their entirety.

While this invention has been particularly shown and described with references to preferred embodiments thereof, it will be understood by those skilled in the art that various changes in form and details may be made therein without departing from the scope of the invention encompassed by the appended claims.

What is claimed is:

1. An isolated nucleic acid molecule comprising a nucleic acid sequence selected from the group consisting of:
a) a nucleic acid sequence of SEQ ID Nos.: 1, 3, 5, 7, 9, 11 or 13;
b) a nucleic acid sequence of the coding region of SEQ ID Nos.: 1, 3, 5, 7, 9, 11 or 13;
c) a complement of a nucleic acid sequence of SEQ ID Nos.: 1, 3, 5, 7, 9, 11 or 13;
d) a complement of a nucleic acid sequence of the coding region of SEQ ID Nos.: 1, 3, 5, 7, 9, 11 or 13;
e) a nucleic acid sequence that encodes SEQ ID Nos. 2, 4, 6, 8, 10, 12, 14 or 15;
f) a nucleic acid sequence that hybridizes under high stringency conditions to the nucleic acid sequence of SEQ ID Nos.: 1, 3, 5, 7, 9, 11 or 13;
g) a nucleic acid sequence that hybridizes under high stringency conditions to the nucleic acid sequence of the coding region of SEQ ID Nos.: 1, 3, 5, 7, 9, 11 or 13;
h) an isolated nucleic acid molecule comprising a nucleic acid sequence having at least about 60% identity with SEQ ID NO.: 1, 3, 5, 7, 9, 11 or 13, or the coding region thereof, and encodes a polypeptide that modulates engulfment of dying cells or particles from dying cells, and cell migration;
i) an isolated nucleic acid molecule comprising a nucleic acid sequence having at least about 70% identity with SEQ ID NO.: 1, 3, 5, 7, 9, 11 or 13, or the coding region thereof; and encodes a polypeptide that modulates engulfment of dying cells or particles from dying cells, and cell migration;
j) an isolated nucleic acid molecule comprising a nucleic acid sequence having at least about 80% identity with SEQ ID NO.: 1, 3, 5, 7, 9, 11 or 13, or the coding region thereof; and encodes a polypeptide that modulates engulfment of dying cells or particles from dying cells, and cell migration; and
k) an isolated nucleic acid molecule comprising a nucleic acid sequence having at least about 90% identity with SEQ ID NO.: 1, 3, 5, 7, 9, 11 or 13, or the coding region thereof; and encodes a polypeptide that modulates engulfment of dying cells or particles from dying cells, and cell migration.

2. A probe comprising a nucleic acid sequence of claim 1.

3. A peptide or protein comprising an amino acid sequence selected from group consisting of:
a) an amino acid sequence of SEQ ID Nos.: 2, 4, 6, 8, 10, 12, 14 or 15;
b) an amino acid sequence of the coding region of SEQ ID Nos.: 2, 4, 6, 8, 10, 12, 14 or 15; and
c) an amino acid sequence encoded by SEQ ID Nos.: 1, 3, 5, 7, 9, 11 or 13.

4. A vector or plasmid comprising a nucleic acid sequence of claim 1.

5. A vector or plasmid that comprises a nucleic acid molecule that encodes an amino acid sequence of claim 3.

6. A cell comprising a nucleic acid sequence of claim 1.

7. A cell that comprises a nucleic acid molecule that encodes an amino acid sequence of claim 3.

8. An antibody or antibody fragment that binds to a portion of a polypeptide molecule having an amino acid sequence of claim 3.

9. An antibody of claim 8, which is a polyclonal antibody.

10. An antibody of claim 8, which is a monoclonal antibody.

11. A fusion protein comprising the peptide or protein of claim 3, and a portion of an immunoglobulin.

12. An antagonist of a peptide or protein having an amino acid sequence of claim 3.

13. An antagonist of a nucleic acid sequence of claim 1.

14. An agonist of a peptide or protein having an amino acid sequence of claim 3.

15. An agonist of a nucleic acid sequence of claim 1.

16. An assay for determining the presence or absence of a peptide or protein having an amino acid sequence of SEQ ID Nos: 2, 4, 6, 8, 10, 12, 14, or 15, in a sample, comprising:
a) contacting a sample to be tested with an antibody specific to a peptide or protein having an amino acid sequences of SEQ ID Nos: 2, 4, 6, 8, 10, 12, 14 or 15, or a fragment thereof sufficiently to allow formation of a complex between the peptide or protein and the antibody, and
b) detecting the presence or absence of the complex formation.

17. A non-human transgenic animal comprising an isolated nucleic acid molecule comprising a nucleic acid sequence of claim 1.

18. A non-human transgenic animal comprising a gene encoding a peptide or protein having an amino acid sequence of claim 3.

19. A transgenic nematode worm comprising a nucleic acid molecule having a nucleic acid sequence of SEQ ID NO.:1, 3, 5, 7, 9, 11 or 13, wherein SEQ ID NO.: 1, 3, 5, 7, 9, 11 or 13 is mutated, deleted or encodes a non-functional polypeptide.

20. A method for determining whether a compound is an enhancer or inhibitor of phagocytosis of dying cells or cell migration, comprising:
a) exposing a transgenic nematode worm of claim 19 to the compound to be tested, and
b) measuring the level of phagocytic activity or cell migration, wherein an increase in the level of phagocytic activity or cell migration indicates an enhancer, and a decrease in the level of phagocytic activity or cell migration indicates an inhibitor.

21. A method for identifying a compound that is an inhibitor or an enhancer of phagocytosis of dying cells, comprising:
a) exposing a transgenic mammalian cell to a compound in the presence of a dying cell, wherein the transgenic mammalian cell comprises a nucleic acid molecule having a nucleic acid sequence of claim 1; and
b) measuring the rate of phagocytic uptake by said transgenic cells; wherein an increased rate of phagocytosis indicates an enhancer and a decreased rate of phagocytosis indicates an inhibitor.

22. A compound identifiable according to the method of claim 21.

23. A method for identifying a compound that is an inhibitor or an enhancer of phagocytosis of dying cells, comprising:
a) exposing a transgenic mammalian cell to a compound in the presence of a dying cell, wherein the transgenic mammalian cell that encodes a peptide or protein having an amino acid sequence of claim 3; and
b) measuring the rate of phagocytic uptake by said transgenic cells; wherein an increased rate of phagocytosis indicates an enhancer and a decreased rate of phagocytosis indicates an inhibitor.

24. A compound identifiable according to the method of claim 23.

25. A method of inhibiting phagocytosis of dying cells or cell migration in an organism, comprising subjecting the organism to a compound that inhibits a peptide or protein comprising an amino acid sequence of claim 3 wherein a decrease of phagocytic activity or cell migration occurs.

26. The method of claim 25, wherein the compound that inhibits the peptide or protein is an antibody or antibody fragment.

27. A method of enhancing phagocytosis of dying cells or cell migration in an organism, comprising subjecting the organism to a compound that enhances a peptide or protein comprising an amino acid sequence of claim 3, wherein an increase of phagocytic activity or cell migration occurs.

28. A method of treating a mammal having a disease involving a defect in ELMO1, ELMO2, ELMO3, or a pathway thereof, comprising administering a compound that comprises:
a) a peptide or protein comprising an amino acid sequence selected from group consisting of:
i) an amino acid sequence of SEQ ID Nos.: 2, 4, 6, 8, 10, 12, 14 or 15;
ii) an amino acid sequence of the coding region of SEQ ID Nos.: 2, 4, 6, 8, 10, 12, 14 or 15; and
iii) an amino acid sequence encoded by SEQ ID Nos.: 1, 3, 5, 7, 9, 11 or 13.
b) a nucleic acid molecule comprising a nucleic acid sequence selected from the group consisting of:
i) a nucleic acid sequence of SEQ ID Nos.: 1, 3, 5, 7, 9, 11 or 13;
ii) a nucleic acid sequence of the coding region of SEQ ID Nos.: 1, 3, 5, 7, 9, 11 or 13;
iii) a complement of a nucleic acid sequence of SEQ ID Nos.: 1, 3, 5, 7, 9, 11 or 13;
iv) a complement of a nucleic acid sequence of the coding region of SEQ ID Nos.: 1, 3, 5, 7, 9, 11 or 13; and
v) a nucleic acid sequence that encodes SEQ ID Nos. 2, 4, 6, 8, 10, 12, 14 or 15.

29. The method of claim 28, wherein inflammation, autoimmune disease or cancer is treated and a increase in phagocytic activity occurs.

30. The method of claim 28, wherein a neurodegenerative disease, stroke or sickle cell anemia is treated and a decrease in phagocytic activity occurs.

31. A method of treating a mammal having a disease involving a defect in ELMO1, ELMO2, or ELMO3, or a pathway thereof, comprising administering the compound of claim 22, wherein an increase or decrease in phagocytotic activity occurs.

32. The method of claim 31, wherein inflammation, autoimmune disease or cancer is treated and a increase in phagocytic activity occurs.

33. The method of claim 31, wherein a neurodegenerative disease, stroke or sickle cell anemia is treated and a decrease in phagocytic activity occurs.
